(19) United States
(12) Patent Application Publication
(10) Pub. No.: US 2012/0135064 A1
(43) Pub. Date: May 31, 2012

(54) CONJUGATED NANODELIVERY VEHICLES

(75) Inventor: Robert B. Campbell, Quincy, MA (US)

(73) Assignee: Northeastern University, Boston, MA (US)

(21) Appl. No.: 13/322,338

(22) PCT Filed: May 26, 2010
(86) PCT No.: PCT/US10/36147

§ 371 Date: Jan. 30, 2012

Related U.S. Application Data
(60) Provisional application No. 61/181,387, filed on May 27, 2009.

(51) Int. Cl. A61K 9/127; G01N 33/574; C12N 5/02; A61K 39/395

(52) U.S. Cl. 424/450; 424/133.1; 435/7.23; 435/375

(57) ABSTRACT

Anti-angiogenesis agent-linked liposomes and micelles, methods of making such liposomes and micelles, and methods of using such liposomes and micelles, such as for delivery of therapeutic and detection agents to tumor cells, are described.

### CROSS-REFERENCE TO RELATED APPLICATIONS

This application claims the benefit of priority to U.S. Provisional Application No. 61/181,387, filed May 27, 2009, the contents of which are incorporated by reference herein in their entirety.

### FIELD OF THE INVENTION

The invention is in the field of nanotechnology including nano-medicine and nano-imaging.

### BACKGROUND OF THE INVENTION

Chemotherapeutic agents for treating solid tumors are known. Conventional cationic (positively-charged) liposomes were initially developed for gene therapy but are now being evaluated in the clinic for their ability to deliver chemotherapeutic drugs to solid tumors. There is still a need for effective treatments for solid tumors that maximize the delivery of drugs to tumor targets while minimizing uptake by healthy tissue.

### SUMMARY OF THE INVENTION

The invention is based, at least in part, on the discovery that an anti-angiogenic agent conjugated to the surface of a liposome enhances targeting and treatment of a tumor.

In one aspect, the invention features a method of detecting a cancer cell in a subject, comprising administering to the subject a liposome or micelle, the liposome or the micelle comprising (i) an anti-angiogenesis agent on an outer surface of the liposome or the micelle, and (ii) a detection agent conjugated to the liposome or the micelle; and detecting the detection agent, thereby detecting the cancer cell.

In some embodiments, the anti-angiogenesis agent is an anti-VEGF antibody. In certain embodiments, the anti-VEGF antibody is bevacizumab.

In some embodiments, the liposome or the micelle comprises a cationic lipid. In particular embodiments, the cationic lipid is DDAB, DODAP, DOTAP, DOTMA, DMTAP, or DSTAP.

In certain embodiments, the cationic lipid comprises a derivatized cationic lipid. In some embodiments, the derivatized cationic lipid comprises polyethylene glycol (PEG).

In some embodiments, about 50% to about 100% of the outer surface of the liposome or the micelle comprises the anti-angiogenesis agent. In other embodiments, about 10%, about 20%, about 30%, about 40%, about 50%, about 60%, about 70%, about 80%, about 90%, or about 100% of the outer surface area of the liposome or the micelle comprises the anti-angiogenesis agent.

In some embodiments, the anti-angiogenesis agent comprises about 20% to about 60% of the liposome or the micelle by weight. In particular embodiments, the anti-angiogenesis agent comprises about 10%, about 20%, about 30%, about 40%, about 50%, about 60%, about 70%, about 80%, or about 90% of the liposome or micelle by weight.

In certain embodiments, the detection agent is a magnetic resonance imaging (MRI) contrast agent, a computed tomography (CT scan) imaging agent, an optical imaging agent, or a radionuclide. In particular embodiments, the radionuclide is iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

In some embodiments, the cancer cell is a squamous cancer cell cancer, lung cancer cell, peritoneum cancer cell, hepatocellular cancer cell, gastrointestinal cancer cell, pancreatic cancer cell, glioblastoma cell, cervical cancer cell, ovarian cancer cell, liver cancer cell, bladder cancer cell, hepatoma cell, breast cancer cell, colon cancer cell, rectal cancer cell, colorectal cancer cell, endometrial cancer cell, uterine carcinoma cell, salivary gland carcinoma cell, kidney or renal cancer cell, prostate cancer cell, vulval cancer cell, thyroid cancer cell, hepatic carcinoma cell, anal carcinoma cell, or penile carcinoma cell.

In certain embodiments, the subject is a human, ape, monkey, orangutan, chimpanzee, dog, cat, guinea pig, rabbit, rat, mouse, horse, cattle, or cow.

In another aspect, the invention features a method of delivering a chemotherapeutic agent to a cancer cell, comprising contacting the cancer cell with a liposome or a micelle, the liposome or the micelle comprising (i) an anti-angiogenesis agent on an outer surface of the liposome or the micelle, and (ii) a chemotherapeutic agent conjugated to the liposome or the micelle, the anti-angiogenesis agent targeting the cancer cell, thereby delivering the chemotherapeutic agent to the cancer cell.

In some embodiments, the anti-angiogenesis agent is an anti-VEGF antibody. In particular embodiments, the anti-VEGF antibody is bevacizumab.

In some embodiments, the liposome or the micelle comprises a cationic lipid. In particular embodiments, the cationic lipid is DDAB, DODAP, DOTAP, DOTMA, DMTAP, or DSTAP.

In some embodiments, the cationic lipid comprises a derivatized cationic lipid. In certain embodiments, the derivatized cationic lipid comprises PEG.

In some embodiments, about 50% to about 100% of the outer surface of the liposome or the micelle comprises the anti-angiogenesis agent. In other embodiments, about 10%, about 20%, about 30%, about 40%, about 50%, about 60%, about 70%, about 80%, about 90%, or about 100% of the outer surface area of the liposome or the micelle comprises the anti-angiogenesis agent.

In some embodiments, the anti-angiogenesis agent comprises about 20% to about 60% of the liposome or the micelle by weight. In particular embodiments, the anti-angiogenesis agent comprises about 10%, about 20%, about 30%, about 40%, about 50%, about 60%, about 70%, about 80%, or about 90% of the liposome or micelle by weight.

In certain embodiments, the chemotherapeutic agent is 6 mercaptopurine, 6-thioguanine, cytarabine, 5-fluorouracil decarbazine, mechlorethamine, thioepa chlorambucil, CC-1065, melphalan, carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, doxorubicin, dactinomycin, bleomycin, mithramycin, anthramycin (AMC), vincristine, vinblastine, taxol, maytansinoids, cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, or calicheamicin.

In some embodiments, the cancer cell is a squamous cancer cell cancer, lung cancer cell, peritoneum cancer cell, hepatocellular cancer cell, gastrointestinal cancer cell, pancreatic cancer cell, glioblastoma cell, cervical cancer cell, ovarian cancer cell, liver cancer cell, bladder cancer cell, hepatoma cell, breast cancer cell, colon cancer cell, rectal cancer cell, colorectal cancer cell, endometrial cancer cell, uterine carcinoma cell, salivary gland carcinoma cell, kidney or renal cancer cell, prostate cancer cell, vulval cancer cell, thyroid cancer cell, hepatic carcinoma cell, anal carcinoma cell, or penile carcinoma cell.

In some embodiments, the cancer cell is in a subject, and the chemotherapeutic agent is administered to the subject. In certain embodiments, the subject is a human, ape, monkey, orangutan, chimpanzee, dog, cat, guinea pig, rabbit, rat, mouse, horse, cattle, or cow. In other embodiments, the chemotherapeutic agent is delivered to the cell in vitro.

In other embodiments, the liposome or micelle further comprises a detection agent, and the method further comprises detecting the detection agent, thereby detecting the cancer cell. In certain embodiments, the detection agent is a magnetic resonance imaging (MRI) contrast agent, a computed tomography (CT scan) imaging agent, an optical imaging agent, or a radionuclide. In particular embodiments, the radionuclide is iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi) indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

In another aspect, the invention features a method of treating a cancer cell in a subject, comprising administering to the subject a cationic liposome or cationic micelle, the cationic liposome or the cationic micelle comprising (i) a cationic lipid; (ii) PEG conjugated to the cationic lipid; (iii) an anti-angiogenesis agent on an outer surface of the cationic liposome or the cationic micelle; and (iv) a chemotherapeutic agent conjugated to the liposome or the micelle, the anti-angiogenesis agent targeting the cancer cell, thereby treating the cancer cell.

In some embodiments, the anti-angiogenesis agent is an anti-VEGF antibody. In particular embodiments, the anti-VEGF antibody is bevacizumab.

In some embodiments, the cationic lipid is DDAB, DODAP, DOTAP, DOTMA, DMTAP, or DSTAP.

In some embodiments, about 50% to about 100% of the outer surface of the liposome or the micelle comprises the anti-angiogenesis agent. In other embodiments, about 10%, about 20%, about 30%, about 40%, about 50%, about 60%, about 70%, about 80%, about 90%, or about 100% of the outer surface area of the liposome or the micelle comprises the anti-angiogenesis agent.

In some embodiments, the anti-angiogenesis agent comprises about 20% to about 60% of the liposome or the micelle by weight. In particular embodiments, the anti-angiogenesis agent comprises about 10%, about 20%, about 30%, about 40%, about 50%, about 60%, about 70%, about 80%, or about 90% of the liposome or micelle by weight.

In certain embodiments, the chemotherapeutic agent is 6 mercaptopurine, 6-thioguanine, cytarabine, 5-fluorouracil decarbazine, mechlorethamine, thioepa chlorambucil, CC-1065, melphalan, carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, doxorubicin, dactinomycin, bleomycin, mithramycin, anthramycin (AMC), vincristine, vinblastine, taxol, maytansinoids, cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, or calicheamicin.

In some embodiments, the cancer cell is a squamous cancer cell cancer, lung cancer cell, peritoneum cancer cell, hepatocellular cancer cell, gastrointestinal cancer cell, pancreatic cancer cell, glioblastoma cell, cervical cancer cell, ovarian cancer cell, liver cancer cell, bladder cancer cell, hepatoma cell, breast cancer cell, colon cancer cell, rectal cancer cell, colorectal cancer cell, endometrial cancer cell, uterine carcinoma cell, salivary gland carcinoma cell, kidney or renal cancer cell, prostate cancer cell, vulval cancer cell, thyroid cancer cell, hepatic carcinoma cell, anal carcinoma cell, or penile carcinoma cell.

In certain embodiments, the subject is a human, ape, monkey, orangutan, chimpanzee, dog, cat, guinea pig, rabbit, rat, mouse, horse, cattle, or cow.

In other embodiments, the liposome or micelle further comprises a detection agent, and the method further comprises detecting the detection agent, thereby detecting the cancer cell. In certain embodiments, the detection agent is a magnetic resonance imaging (MRI) contrast agent, a computed tomography (CT scan) imaging agent, an optical imaging agent, or a radionuclide. In particular embodiments, the radionuclide is iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine cum, rhenium (¹⁸⁶Re or ¹⁸⁷Re), bismuth (²¹²Bi or ²¹³Bi), indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

In another aspect, the invention features a method of treating a disease or disorder described herein, comprising administering to a subject in need thereof a cationic liposome or cationic micelle, the cationic liposome or the cationic micelle comprising (i) a cationic lipid; (ii) PEG conjugated to the cationic lipid; (iii) an anti-angiogenesis agent on an outer surface of the cationic liposome or the cationic micelle; and (iv) a chemotherapeutic agent conjugated to the liposome or the micelle, the anti-angiogenesis agent targeting a cancer cell, thereby treating the disease or disorder.

In some embodiments, the anti-angiogenesis agent is an anti-VEGF antibody. In particular embodiments, the anti-VEGF antibody is bevacizumab.

In some embodiments, the cationic lipid is DDAB, DODAP, DOTAP, DOTMA, DMTAP, or DSTAP.

In some embodiments, about 50% to about 100% of the outer surface of the liposome or the micelle comprises the anti-angiogenesis agent. In other embodiments, about 10%, about 20%, about 30%, about 40%, about 50%, about 60%, about 70%, about 80%, about 90%, or about 100% of the outer surface area of the liposome or the micelle comprises the anti-angiogenesis agent.

In some embodiments, the anti-angiogenesis agent comprises about 20% to about 60% of the liposome or the micelle by weight. In particular embodiments, the anti-angiogenesis agent comprises about 10%, about 20%, about 30%, about 40%, about 50%, about 60%, about 70%, about 80%, or about 90% of the liposome or micelle by weight.

In certain embodiments, the chemotherapeutic agent is 6 mercaptopurine, 6-thioguanine, cytarabine, 5-fluorouracil decarbazine, mechlorethamine, thioepa chlorambucil, CC-1065, melphalan, carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, doxorubicin, dactinomycin, bleomycin, mithramycin, anthramycin (AMC), vincristine, vinblastine, taxol, maytansinoids, cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, or calicheamicin.

In certain embodiments, the subject is a human, ape, monkey, orangutan, chimpanzee, dog, cat, guinea pig, rabbit, rat, mouse, horse, cattle, or cow.

In other embodiments, the liposome or micelle further comprises a detection agent, and the method further comprises detecting the detection agent, thereby detecting a cancer cell. In certain embodiments, the detection agent is a magnetic resonance imaging (MRI) contrast agent, a computed tomography (CT scan) imaging agent, an optical imaging agent, or a radionuclide. In particular embodiments, the radionuclide is iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium (¹¹¹In), technetium (^{99m}Tc), phosphours (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

In another aspect, the invention features a conjugated liposome or micelle described herein. In some embodiments, the liposome or micelle is conjugated to an anti-angiogenic agent, e.g., an anti-VEGF antibody, e.g., bevacizumab. In some embodiments, the liposome or micelle comprises a cationic lipid, e.g., DDAB, DODAP, DOTAP, DOTMA, DMTAP, or DSTAP. In some embodiments, the liposome or micelle comprises a derivatized cationic lipid, e.g., derivatized with PEG. In some embodiments, the conjugated liposome or micelle binds to a soluble angiogenic agent, e.g., binds to soluble VEGF, e.g., binds to soluble VEGF in the blood.

In some embodiments, the liposome or micelle comprises an anti-VEGF antibody, e.g., bevacizumab, wherein the anti-VEGF antibody specifically binds soluble VEGF in the blood. In some embodiments, the bound VEGF specifically binds to a VEGF receptor on a tumor, targeting the liposome or micelle to the tumor.

In some embodiments, the liposome or micelle further comprises a chemotherapeutic agent conjugated to the liposome or the micelle. In certain embodiments, the chemotherapeutic agent is 6 mercaptopurine, 6-thioguanine, cytarabine, 5-fluorouracil decarbazine, mechlorethamine, thioepa chlorambucil, CC-1065, melphalan, carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, doxorubicin, dactinomycin, bleomycin, mithramycin, anthramycin (AMC), vincristine, vinblastine, taxol, maytansinoids, cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, or calicheamicin.

In other embodiments, the liposome or micelle further comprises a detection agent. In certain embodiments, the detection agent is a magnetic resonance imaging (MRI) contrast agent, a computed tomography (CT scan) imaging agent, an optical imaging agent, or a radionuclide. In particular embodiments, the radionuclide is iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

The following figures are presented for the purpose of illustration only, and are not intended to be limiting.

### BRIEF DESCRIPTION OF THE DRAWINGS

FIG. 1A is a graphic representation of cell growth and VEGF secretion of various pancreatic and endothelial cells (Capan-1, HPAF-II, PANC-1, MS1-VEGF and HMEC-1 cells) after 24 hours, 48 hours, 72 hours, 96 hours, 120 hours, and 144 hours in culture.

FIG. 1B is a graphic representation of cell growth and VEGF secretion of various pancreatic and endothelial cells (Capan-1, HPAF-II, PANC-1, MS1-VEGF, and HMEC-1) grown with or without VEGF in the media, where the amount of VEGF secreted or present in the media was determined through an ELISA at 6 time points represented by raw absorbance values.

FIG. 2 is a graphic representation of bevacizumab toxicity on Capan-1, HPAF-II, PANC-1, MS1-VEGF and HMEC-1 cells grown in media containing or free of VEGF after a 24 h exposure.

FIG. 3A is a graphic representation of bevacizumab modified and unmodified PEGylated cationic liposome (“PCL”) toxicity on Capan-1 cells grown in media containing or free of VEGF, showing that all cell lines were relatively non-toxic to the effects of either liposome formulation up to 100 nmoles (*—p<0.05 and #—p<0.01 than the untreated control, where the symbols show how significant one experimental group is when compared to another).

FIG. 3B is a graphic representation of bevacizumab modified and unmodified PCL toxicity on HPAF-II, cells grown in media containing or free of VEGF, showing that all cell lines were relatively non-toxic to the effects of either liposome formulation up to 100 nmoles (*—p<0.05 and #—p<0.01 than the untreated control).

FIG. 3C is a graphic representation of bevacizumab modified and unmodified PCL toxicity on, PANC-1 cells grown in media containing or free of VEGF, showing that all cell lines were relatively non-toxic to the effects of either liposome formulation up to 100 nmoles (*—p<0.05 and #—p<0.01 than the untreated control).

FIG. 3D is a graphic representation of bevacizumab modified and unmodified PCL toxicity on, MS1-VEGF cells grown in media containing or free of VEGF, showing that all cell lines were relatively non-toxic to the effects of either liposome formulation up to 100 nmoles (*—p<0.05 and #—p<0.01 than the untreated control).

FIG. 3E-3F are graphic representations of bevacizumab modified and unmodified PCL toxicity on HMEC-1 cells grown in media containing (E), or free (F), of VEGF, showing that all cell lines ere relatively non-toxic to the effects of either liposome formulation up to 100 nmoles (*—p<0.05 and #—p<0.01 than the untreated control).

FIG. 4 is a graphic representation of cell association of bevacizumab-modified (white bar) and unmodified (black bar) PCLs with Capan-1, MS1-VEGF and HMEC-1 cells grown in media containing VEGF.

FIGS. 5A-5L are graphic representations of flow cytometer analyses of cell association of fluorescein-labeled bevacizumab modified and unmodified cationic and electroneutral liposomes with Capan-1, HPAF-II, PANC-1, MS1-VEGF and HMEC-1 cells grown in media containing or free of VEGF after 1 hour incubation. FIGS. 5A-5F represent cationic liposomes and FIGS. 5G-5L represent electroneutral liposomes. FIGS. 5A and 5G are CAPAN-1 cells, FIGS. 5B and 5H are HPAF-II cells, FIGS. 5C and 5I are PANC-1 cells, FIGS. 5D and 5J are MS1-VEGF cells, FIGS. 5E and 5K are HMEC-1 cells grown with VEGF, FIGS. 5F and 5L are HMEC-1 cells grown without VEGF. The dotted line is untreated cells, thin line is unmodified liposomes, and thick line is bevacizumab modified liposomes.

FIG. 6 is a collection of representations of fluorescence micrographs of cells after incubation with fluorescein labeled bevacizumab modified and unmodified PCLs showing Capan-1, HPAF-II, PANC-1, MS1-VEGF and HMEC-1 cells grown in media containing or free of VEGF and incubated with 100 μmole of liposomes. The gray scale picture was taken with a DIC microscope and the white in the white on black picture represents the fluorescein fluorescence. Scale bar measures 20 μm.

FIG. 7A is a graphic representation of the biodistribution of the bevacizumab modified and unmodified liposomes in CAPAN-1 tumor-bearing mice. The biodistribution was evaluated after 24 hours and shows the percent of injected dose per gram of tissue, organ biodistribution (Inset—blood distribution).

FIG. 7B is a graphic representation of the biodistribution of the bevacizumab modified and unmodified liposomes in CAPAN-1 tumor bearing mice. The biodistribution in the tumor was evaluated after 24 hours.

FIG. 8A is a graphic representation of percent change in bodyweight of untreated HPAF-II tumor bearing mice.

FIG. 8B is a graphic representation of percent change in bodyweight of HPAF-II tumor bearing mice treated with bevacizumab alone on days 1, 4, 7, and 10.

FIG. 8C is a graphic representation of percent change in bodyweight of HPAF-II tumor bearing mice treated with unmodified PCLs on days 1, 4, 7, and 10.

FIG. 8D is a graphic representation of percent change in bodyweight of HPAF-II tumor bearing mice treated with bevacizumab-modified PCLs on days 1, 4, 7, and 10.

FIG. 9 is a graphic representation of mean tumor volume in untreated HPAF-II tumor bearing mice, in mice treated with unmodified PCLs, in mice treated with bevacizumab alone, or in mice treated with bevacizumab-modified PCLs. Arrows represent injection days. Bars represent standard errors using the following symbols: “*”=compared to untreated control; “#”=compared to unmodified PCLs; and “&”=compared bevacizumab-modified PCLs. For “*”, “#”, and “&”, p≦0.05; for “**”, “##”, and “&&”, p≦0.01; for “***”, “###”, and “&&&”, p≦0.001, using t-tests.

FIG. 10A is a graphic representation of percent change in bodyweight of untreated Capan-1 tumor bearing mice.

FIG. 10B is a graphic representation of percent change in bodyweight of Capan-1 tumor bearing mice treated with unmodified PCLs on days 1, 4, 7, and 10.

FIG. 10C is a graphic representation of percent change in bodyweight of Capan-1 tumor bearing mice treated with bevacizumab-modified PCLs on days 1, 4, 7, and 10.

FIG. 11 is a graphic representation of mean tumor volume in untreated Capan-1 tumor bearing mice, in mice treated with unmodified PCLs, and in mice treated with bevacizumab-modified PCLs. Arrows represent injection days. Error bars represent standard errors using the following symbols: “*”=compared to untreated control and “#”=compared to unmodified PCLs. For “*” and “#”, p≦0.05; for “**” and “##”, p≦0.01; and for “***” and “###”, p≦0.001, using ANOVA with Tukey post-hoc testing.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DEFINITIONS

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “about” is used herein to mean a value −or +20% of a given numerical value. Thus, “about 60%” means a value of between 60−(20% of 60) and 60+(20% of 60) (i.e., between 48 and 70).

The terms “peptide”, “polypeptide” and “protein” are used interchangeably herein.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

The term “treatment” or “treating”, as used herein, refers to administering a therapy in an amount, manner, and/or mode effective to improve a condition, symptom, or parameter associated with a disorder or condition or to prevent or reduce progression of a disorder or condition, either to a statistically significant degree or to a degree detectable to one skilled in the art. An effective amount, manner, or mode can vary depending on the subject and may be tailored to the subject.

The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda. In one embodiment, the antibody is glycosylated.

As used herein, the terms “coupled”, “linked”, “fused”, and “fusion” are used interchangeably. These terms refer to the joining together of two more elements or components by whatever means, including chemical conjugation or recombinant means.

The term “drug,” as used herein, refers to any substance used in the prevention, diagnosis, alleviation, treatment, or cure of a disease or condition.

As used herein, the terms “anti-angiogenesis agent” and “anti-angiogenic agent” refer to any compound or substance that inhibits or discourages angiogenesis, whether alone or in combination with another substance.

### General

The disclosure is based, in part, on the discovery that an anti-angiogenic agent conjugated to the surface of a liposome enhances the targeting of the liposome to a tumor as well as its treatment with the anti-angiogenic agent. The anti-angiogenic agent can also mediate targeted delivery of an additional therapeutic agent, e.g., a chemotherapeutic agent encapsulated within or conjugated to a liposome, to a tumor. In certain instances, the anti-angiogenic agent and the chemotherapeutic agent exhibit synergistic activity, e.g., synergistic therapeutic activity. The liposome can also be conjugated to a detection agent, and the anti-angiogenic agent can mediate detection or imaging of a tumor.

### Liposomes

Liposomes are vesicles that include one or more concentrically ordered lipid bilayer(s) encapsulating an aqueous phase, when in an aqueous environment. Such vesicles are formed in the presence of “vesicle-forming lipids”, which are defined herein as amphipathic lipids capable of either forming or being incorporated into a bilayer structure. The term includes lipids that are capable of forming a bilayer by themselves or when in combination with another lipid or lipids. An amphipathic lipid is incorporated into a lipid bilayer by having its hydrophobic moiety in contact with the interior, hydrophobic region of the bilayer membrane and its polar head moiety oriented towards an outer, polar surface of the membrane. Hydrophilicity arises from the presence of functional groups, such as hydroxyl, phosphate, carboxyl, sulfate, amino or sulfhydryl groups. Hydrophobicity results from the presence of a long chain of aliphatic hydrocarbon groups.

Liposomes include multilamellar vesicles, multivesicular liposomes, unilamellar vesicles, and giant liposomes. Multilamellar liposomes (also known as multilamellar vesicles (“MLV”)) contain multiple concentric bilayers within each liposome particle, resembling the layers of an onion. Multivesicular liposomes consist of lipid membranes enclosing multiple non-concentric aqueous chambers. Unilamellar liposomes enclose a single internal aqueous compartment. Single bilayer (or substantially single bilayer) liposomes include small unilamellar vesicles (“SUV”) and large unilamellar vesicles (“LUV”). LUVs and SUVs can range in size from about 50 nm to about 500 nm and about 20 nm to about 50 nm, respectively. Giant liposomes can range in size from about 5000 nm to about 50,000 nm (Needham et al., Colloids and Surfaces B: Biointerfaces 18:183-195 (2000)).

Any suitable vesicle-forming lipid (e.g., naturally occurring lipids and synthetic lipids) can be utilized in the liposomes and micelles described herein. Suitable lipids include, without limitation, phospholipids such as phosphatidylcholine (PC), phosphatidylglycerol (PG), phosphatidylinositol (PI), phosphatidic acid (PA), phosphatidyethanolamine (PE), phosphatidylserine (PS), and phosphoethanolamine; sterols such as cholesterol; glycolipids; sphingolipids such as sphingosine, ceramides, sphingomyelin, and glycosphingolipids (such as cerebrosides and gangliosides). Particular lipids include dipalmitoyl phosphatidylcholine, cholesterol, ganglioside, dicetyl phosphate, dipalmitoyl phosphatidylethanolamine, sodium cholate, dicetyl phosphatidylethanolamine-polyglycerin 8G, dimyristoyl phosphatidylcholine, distearoyl phosphatidylcholine, dioleoyl phosphatidylcholine, dimyristoyl phosphatidylserine, dipalmitoyl phosphatidylserine, distearoyl phosphatidylserine, dioleoyl phosphatidylserine, dimyristoyl phosphatidylinositol, dipalmitoyl phosphatidylinositol, distearoyl phosphatidylinositol, dioleoyl phosphatidylinositol, dimyristoyl phosphatidylethanolamine, distearoyl phosphatidylethanolamine, distearoyl phosphoethanolamine, dioleoyl phosphatidylethanolamine, dimyristoyl phosphatidic acid, dipalmitoyl phosphatidic acid, distearoyl phosphatidic acid, dioleoyl phosphatidic acid, galactosyl ceramides, glycosyl ceramides, lactosyl ceramides, phosphatides, globosides, GM1 (Galβ1, 3GalNAcβ1, 4(NeuAa-2,3)Galβ1, 4Glcβ1, 1'Cer), ganglioside GD1a, ganglioside GD1b, dimyristoyl phosphatidylglycerol, dipalmitoyl phosphatidylglycerol, distearoyl phosphatidylglycerol, dioleoyl phosphatidylglycerol, distearoyl-glycero-phosphoethanolamine, and 1,2-dioleoyl-sn-glycero-3-phsophoethanolamine. Suitable phospholipids can include one or two acyl chains having any number of carbon atoms, such as about 6 to about 24 carbon atoms, selected independently of one another and with varying degrees of unsaturation. Thus, combinations of phospholipid of different species and different chain lengths in varying ratios can be used. Mixtures of lipids in suitable ratios, as judged by one of skill in the art, can also be used.

Particular phospholipids useful in the methods described herein are N-[1-(2,3-Dioleoyloxy)propyl]-N,N,N-trimethylammonium methylsulfate (DOTAP), Dimethyldioctadecylammonium (DDAB), 1,2-dioleoyl-3-dimethylammonium-propane (DODAP), 1,2-di-O-octadecenyl-3-trimethylammonium propane (DOTMA), 1,2-Dimyristoyl-3-TrimethylAmmoniumPropane (DMTAP), 1,2-distearyl-3-trimethylammonium propane (DSTAP) or any other known cationic lipid (e.g., such as those available from Avanti Polar Lipids, Alabaster, Ala.).

Liposomes can be generated using a variety of techniques known in the art. These techniques include, without limitation, ether injection (Deamer et al., Acad. Sci. 308:250 (1978)); surfactant (Brunner et al., Biochim. Biophys. Acta 455:322 (1976)); Ca²⁺ fusion (Paphadjopoulos et al., Biochim. Biophys. Acta 394:483 (1975)); freeze-thaw (Pick et al., Arach. Biochim. Biophys. 212:186 (1981)); reverse-phase evaporation (Szoka et al., Biochim. Biophys. Acta 601:559 (1980)); ultrasonic treatment (Huang et al., Biochem. 8:344 (1969)); ethanol injection (Kremer et al., Biochem. 16:3932 (1977)); extrusion (Hope et al., Biochim. Biophys. Acta 812:55 (1985)); French press (Barenholz et al., FEBS Lett. 99:210 (1979)); thin film hydration (Bangham et al., J. Mol. Biol. 13:238-252 (1965)); and any other methods described herein or known in the art. Liposomes can also be generated using commercially available kits (e.g., from Boehringer-Mannheim, ProMega, and Life Technologies (Gibco)).

Different techniques can be used depending on the type of liposome desired. For example, small unilamellar vesicles (SUVs) can be prepared by the ultrasonic treatment method, the ethanol injection method, or the French press method, while multilamellar vesicles (MLVs) can be prepared by the reverse-phase evaporation method or by the simple addition of water to a lipid film, followed by dispersal by mechanical agitation (Bangham et al., J. Mol. Biol. 13:238-252 (1965)). LUVs can be prepared by the ether injection method, the surfactant method, the Ca²⁺ fusion method, the freeze-thaw method, the reverse-phase evaporation method, the French press method, or the extrusion method.

Average liposome size can be determined by known techniques, such as quasi-elastic light scattering, photon correlation spectroscopy, dynamic light scattering, or various electron microscopy techniques (such as negative staining transmission electron microscopy, freeze fracture electron microscopy or cryo-transmission electron microscopy). In some instances, the resulting liposomes can be run down a Sephadex™ G50 column or similar size exclusion chromatography column equilibrated with an appropriate buffer in order to remove unencapsulated therapeutic agents or detection agents described herein.

Liposomes can range in size, such as from about 50 nm to about 1 μm in diameter. For example, liposomes described herein can be less than about 200 nm in diameter, less than about 160 nm in diameter, or less than about 140 nm in diameter. In some embodiments, liposomes described herein can be substantially uniform in size, for example, 10% to 100%, or more generally at least 10%, 20%, 30%, 40%, 50, 55% or 60%, or at least 65%, 75%, 80%, 85%, 90%, or 95%, or as much as 96%, 97%, 98%, 99%, or 100% of the liposomes can have the same size. In some instances, liposomes can be sized by extrusion through a filter (e.g., a polycarbonate filter) having pores or passages of the desired diameter.

In some instances, liposomes can include a hydrophilic moiety. Attaching a hydrophilic moiety to the surface of liposomes can sterically stabilize liposomes and can increase the circulation longevity of the liposome. This can enhance blood stability and increase circulation time, reduce uptake into healthy tissues, and increase delivery to disease sites such as solid tumors (see, e.g., U.S. Pat. Nos. 5,013,556 and 5,593,622; and Patel et al., Crit. Rev. Ther. Drug Carrier Syst. 9:39 (1992)). The hydrophilic moiety can be conjugated to a lipid component of the liposome, forming a hydrophilic polymer-lipid conjugate. The term “hydrophilic polymer-lipid conjugate”, as used herein, refers to a lipid (e.g., a vesicle-forming lipid) covalently joined at its polar head moiety to a hydrophilic polymer, and can be made by attaching the polymer to a reactive functional group at the polar head moiety of the lipid. The covalent linkage can be releasable, such that the polymer dissociates from the lipid (at, e.g., physiological pH or after a variable length of time (see, e.g., Adlakha-Hutcheon et al., Nat. Biotechnol. 17:775-779 (1999)). Nonlimiting suitable reactive functional groups include, e.g., amino, hydroxyl, carboxyl, and formyl groups. The lipid can be any lipid described in the art for use in such conjugates. For example, the lipid can be a phospholipid having one or two acyl chains including between about 6 to about 24 carbon atoms in length with varying degrees of unsaturation.

In some circumstances, the lipid in the conjugate can be a phosphatidyethanolamine, such as of the distearoyl form. The polymer can be a biocompatible polymer. In some instances, the polymer has a solubility in water that permits polymer chains to extend away from a liposome surface with sufficient flexibility that produces uniform surface coverage of a liposome. Such a polymer can be a polyalkylether, including PEG, polymethylene glycol, polyhydroxy propylene glycol, polypropylene glycol, polylactic acid, polyglycolic acid, polyacrylic acid and copolymers thereof, as well as those disclosed in U.S. Pat. Nos. 5,013,556 and 5,395,619. The polymer can have an average molecular weight between about 350 daltons and about 10,000 daltons.

In some instances, the phospholipids can be derivatized phospholipids, such as a PEG-modified phospholipid. The average molecular weight of the PEG can be about 200 daltons to about 20,000 daltons. The liposomes described herein can also be composed of combinations of PEG phospholipids of different species and different chain lengths in varying ratios. Combinations of phospholipids and PEG phospholipids can also be used in forming the liposomes described herein. The derivatized phospholipid can be prepared to include a releasable lipid-polymer linkage such as a peptide, ester, or disulfide linkage.

### Micelles

Micelles are vesicles that include a single lipid monolayer encapsulating an aqueous phase. Micelles can be spherical or tubular and form spontaneously about the critical micelle concentration (“CMC”). In general, micelles are in equilibrium with the monomers under a given set of physical conditions such as temperature, ionic environment, concentration, etc.

Micelles are formed in the presence of “micelle-forming compounds”, which include amphipathic lipids (e.g., a vesicle-forming lipid as described herein or known in the art), lipoproteins, detergents, non-lipid polymers, or any other compound capable of either forming or being incorporated into a monolayer vesicle structure. Thus, a micelle-forming compound includes compounds that are capable of forming a monolayer by themselves or when in combination with another compound, and may be polymer micelles, block co-polymer micelles, polymer-lipid mixed micelles, or lipid micelles. A micelle-forming compound, in an aqueous environment, generally has a hydrophobic moiety in contact with the interior of the vesicle, and a polar head moiety oriented outwards into the aqueous environment. Hydrophilicity generally arises from the presence of functional groups, such as hydroxyl, phosphate, carboxyl, sulfate, amino or sulfhydryl groups. Hydrophobicity generally results from the presence of a long chain of aliphatic hydrocarbon groups.

A micelle can be prepared, e.g., from lipoproteins or artificial lipoproteins including low density lipoproteins, chylomicrons and high density lipoproteins. Micelles can be generated using a variety of known techniques, including, without limitation, simple dispersion by mixing in aqueous or hydroalcoholic media or media containing surfactants or ionic substances; sonication; solvent dispersion; or any other technique described herein or known in the art. Different techniques can be used, depending on the type of micelle desired and the physicochemical properties of the micelle-forming components, such as solubility, hydrophobicity and behavior in ionic or surfactant-containing solutions.

Micelles can range in size, such as between about 5 nm to about 50 nm in diameter. In some instances, micelles can be less than about 50 nm in diameter, less than about 30 nm in diameter, or less than about 20 nm in diameter.

In some situations, micelles described herein can include a hydrophilic polymer-lipid conjugate, as described herein or known in the art.

### Anti-Angiogenic Agents

The methods described herein involve liposomes and/or micelles conjugated to an anti-angiogenic agent, such as a VEGF-specific inhibitor.

An “angiogenic factor or agent” is a growth factor that stimulates the development of blood vessels, e.g., promotes angiogenesis, endothelial cell growth, stability of blood vessels, and/or vasculogenesis. For example, angiogenic factors include, but are not limited to, e.g., VEGF and members of the VEGF family, PlGF, PDGF family, fibroblast growth factor family (FGFs), TIE ligands (Angiopoietins), ephrins, ANGPTL3, and ANGPTL4. Also included are factors that accelerate wound healing, such as growth hormone, insulin-like growth factor-I (IGF-I), VIGF, epidermal growth factor (EGF), CTGF and members of its family, and TGF-alpha and TGF-beta (see, e.g., Klagsbrun et al., Annu. Rev. Physiol. 53:217-39 (1991); Streit et al., Oncogene 22:3172-3179 (2003); Ferrara et al., Nature Medicine 5:1359-1364 (1999); Tonini et al., Oncogene 22:6549-6556 (2003); and Sato, Int. J. Clin. Oncol. 8:200-206 (2003)).

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. For example, an anti-angiogenesis agent can be an antibody or other antagonist to an angiogenic agent, e.g., an antibody to VEGF, an antibody to a VEGF receptor, and a small molecule that blocks VEGF receptor signaling (e.g., PTK787/ZK2284, SU6668, SUTENT/SU11248 (sunitinib malate), and AMG706). Anti-angiogenesis agents also include native angiogenesis inhibitors, e.g., angiostatin and endostatin (see, e.g., Klagsbrun et al., Annu. Rev. Physiol. 53:217-39 (1991); Streit et al., Oncogene 22:3172-3179 (2003); Ferrara et al., Nature Medicine 5:1359-1364 (1999); Tonini et al., Oncogene 22:6549-6556 (2003); and Sato, Int. J. Clin. Oncol. 8:200-206 (2003)).

The anti-angiogenesis agent is conjugated to the liposome or micelle in any way that does not compromise its ability to target a tumor and to treat it. For example, attachment can be performed through standard covalent binding to free amine groups (see, e.g., Torchilin et al., Hybridoma 6:229-240 (1987); Torchilin et al, Biochim. Biophys. Acta 1511:397-411 (2001); Masuko et al., Biomacromol. 6:800-884 (2005)).

In certain instances, an anti-angiogenesis agent is an anti-VEGF neutralizing antibody (or fragment) and/or another VEGF antagonist or a VEGF receptor antagonist including, but not limited to, for example, a soluble VEGF receptor fragment (e.g., VEGFR-1, VEGFR-2, VEGFR-3), a neuropilin fragment (e.g., NRP1, NRP2), an aptamer capable of blocking VEGF or VEGFR, a neutralizing anti-VEGFR antibody, a low molecule weight inhibitor of VEGFR tyrosine kinase (RTK), an antisense molecule for VEGF, a ribozyme against VEGF or VEGF receptors, an antagonist variant of VEGF; and any combination thereof.

In particular instances, the anti-angiogenesis agent is the anti-VEGF antibody bevacizumab (Avastin®, available from Roche, Basel, Switzerland).

In some instances, the anti-angiogenic agent specifically binds to a soluble angiogenic agent, e.g., a soluble angiogenic agent in the blood. For example, an anti-VEGF antibody conjugated to an outer surface of a liposome or micelle can specifically bind soluble VEGF in the blood. Without wishing to be bound by theory, a VEGF/anti-VEGF antibody/liposome complex can be targeted to a tumor by binding to a VEGF receptor on a tumor.

### Detection Agents

In some instances, the liposomes or micelles described herein can be used to detect or image cells, e.g., using a liposome or micelle that includes a detection agent. The detection agent can be used to qualitatively or quantitatively analyze the location and/or the amount of a liposome or micelle at a particular locus. The detection agent can also be used to image a liposome, micelle, and/or a cell or tissue target of a liposome or micelle using standard methods.

A liposome or micelle described herein can be derivatized (or labeled) with a detection agent. Nonlimiting examples of detection agents include, without limitation, fluorescent compounds, various enzymes, prosthetic groups, luminescent materials, bioluminescent materials, fluorescent emitting metal atoms, (e.g., europium (Eu)), radioactive isotopes (described below), quantum dots, electron-dense reagents, and haptens. The detection reagent can be detected using various means including, but are not limited to, spectroscopic, photochemical, radiochemical, biochemical, immunochemical, or chemical means.

Nonlimiting exemplary fluorescent detection agents include fluorescein, fluorescein isothiocyanate, rhodamine, 5-dimethylamine-1-napthalenesulfonyl chloride, phycoerythrin, and the like. A detection agent can also be a detectable enzyme, such as alkaline phosphatase, horseradish peroxidase, β-galactosidase, acetylcholinesterase, glucose oxidase and the like. When a liposome or micelle is derivatized with a detectable enzyme, it can be detected by adding additional reagents that the enzyme uses to produce a detectable reaction product. For example, when the detection agent is horseradish peroxidase, the addition of hydrogen peroxide and diaminobenzidine leads to a detectable colored reaction product. A liposome or micelle can also be derivatized with a prosthetic group (e.g., streptavidin/biotin and avidin/biotin). For example, a liposome or micelle can be derivatized with biotin and detected through indirect measurement of avidin or streptavidin binding. Nonlimiting examples of fluorescent compounds tat can be used as detection reagents include umbelliferone, fluorescein, fluorescein isothiocyanate, rhodamine, dichlorotriazinylamine fluorescein, dansyl chloride, and phycoerythrin. Luminescent materials include, e.g., luminol, and bioluminescent materials include, e.g., luciferase, luciferin, and aequorin.

A detection agent can also be a radioactive isotope, such as, but not limited to, α-, β-, or γ-emitters; or β- and γ-emitters. Radioactive isotopes can be used in diagnostic or therapeutic applications. Such radioactive isotopes include, but are not limited to, iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and gallium (⁶⁷Ga).

The liposomes or micelles can be radiolabeled using techniques known in the art. In some situations, a liposome or micelle described herein is contacted with a chelating agent, e.g., 1,4,7,10-tetraazacyclododecane-N,N′,N″,N′″-tetraacetic acid (DOTA), to thereby produce a conjugated liposome or micelle. The conjugated liposome or micelle is then radiolabeled with a radioisotope, e.g., ¹¹¹In, ⁹⁰Y, ¹⁷⁷Lu, ¹⁸⁶Re, ¹⁸⁷Re, or ^{99m}Tc, to thereby produce a labeled liposome or micelle. In other methods, the liposome or micelle can be labeled with ¹¹¹In and ⁹⁰Y using weak transchelators such as citrate (see, e.g., Khaw et al., Science 209:295-297 (1980)) or ^{99m}Tc after reduction in reducing agents such as Na Dithionite (see, e.g., Khaw et al., J. Nucl. Med. 23:1011-1019 (1982)) or by SnCl₂ reduction (see, e.g., Khaw et al., J. Nucl. Med. 47:868-876 (2006)). Other methods are described in, e.g., Lindegren et al., Bioconjug. Chem. 13:502-509 (2002); Boyd et al., Mol. Pharm. 3:614-627 (2006); and del Rosario et al., J. Nucl. Med. 34:1147-1151 (1993).

### Therapeutic Agents

A liposome or micelle described herein, in addition to an anti-angiogenesis agent described herein, can also include a therapeutic agent. Such liposomes or micelles containing a therapeutic agent can be prepared by conventional active or passive loading methods. For example, a therapeutic agent can be mixed with vesicle-forming lipids and be incorporated within a lipid film, such that when the liposome is generated, the therapeutic agent is incorporated or encapsulated into the liposome. Thus, if the therapeutic agent is substantially hydrophobic, it will be encapsulated in the bilayer of the liposome. Alternatively, if the therapeutic agent is substantially hydrophilic, it will be encapsulated in the aqueous interior of the liposome. The therapeutic agent can be soluble in aqueous buffer or aided with the use of detergents or ethanol. The liposomes can subsequently be purified, for example, through column chromatography or dialysis to remove any unincorporated therapeutic agent.

In some instances, the therapeutic agent can be a therapeutically active radioisotope described above. In other instances, the therapeutic agent is a chemotherapeutic agent.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (such as bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (such as cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimnustine; antibiotics such as enediyne antibiotics (e.g., calicheamicin, calicheamicin gammalI and calicheamicin omegalI (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, and mitomycins (such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin); anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (such as T-2 toxin, verracurin A, roridin A and anguidine); urethan; vindesine (ELDISINEL® and FILDESIN®); dacarbazine; mannomustine; mitobronitol; mitolactol; pipobroman; gacytosine; arabinoside (“Ara-C”); thiotepa; toxoids (e.g., paclitaxel (TAXOL®), albumin-engineered nanoparticle formulations of paclitaxel (ABRAXANE™), and doxetaxel (TAXOTERE®)); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylomithine (DMFO); retinoids such as retinoic acid; pharmaceutically acceptable salts, acids or derivatives of any of the above; as well as combinations of two or more of the above such as CHOP (an abbreviation for a combined therapy of cyclophosphamide, doxorubicin, vincristine, and prednisolone) and FOLFOX (an abbreviation for a treatment regimen with oxaliplatin (ELOXATIN™) combined with 5-FU and leucovovin.

Other chemotherapeutic agents include anti-hormonal agents that act to regulate, reduce, block, or inhibit the effects of hormones that can promote the growth of cancer, and in some cases can be hormones themselves. Nonlimiting examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®).

In addition, chemotherapeutic agents also include bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, such as those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

### Targeting Agents

An outer surface of a liposome or micelle of the disclosure can include, in addition to an anti-angiogenesis agent described herein, an additional targeting agent. The targeting agents can be, for example, various specific ligands, such as antibodies, monoclonal antibodies and their fragments, folate, mannose, galactose and other mono-, di-, and oligosaccharides, and RGD peptide.

The liposomes and micelles described herein are not limited to any particular targeting agent, and a variety of targeting agents can be used. Examples of such targeting agents include, but are not limited to, nucleic acids (e.g., RNA and DNA), polypeptides (e.g., receptor ligands, signal peptides, avidin, Protein A, and antigen binding proteins), polysaccharides, biotin, hydrophobic groups, hydrophilic groups, drugs, and any organic molecules that bind to receptors. In some instances, a liposome or micelle described herein can be conjugated to one, two, or more of a variety of targeting agents. For example, when two or more targeting agents are used, the targeting agents can be similar or dissimilar. Utilization of more than one targeting agent on a particular liposome or micelle can allow the targeting of multiple biological targets or can increase the affinity for a particular target.

The targeting agents can be associated with the liposomes or micelles in a number of ways. For example, the targeting agents can be associated (e.g., covalently or noncovalently bound) to a phospholipid of the liposome or micelle with either short (e.g., direct coupling), medium (e.g., using small-molecule bifunctional linkers such as SPDP (Pierce Biotechnology, Inc., Rockford, Ill.)), or long (e.g., PEG bifunctional linkers (Nektar Therapeutics, Inc., San Carlos, Calif.)) linkages.

In addition, a liposome or micelle can also incorporate reactive groups (e.g., amine groups such as polylysine, dextranemine, profamine sulfate, and/or chitosan). The reactive group can allow for further attachment of various specific ligands or reporter groups (e.g., ¹²⁵I, ¹³¹I, I, Br, various chelating groups such as DTPA, which can be loaded with reporter heavy metals such as ¹¹¹In, ^{99m}Tc, Gd, Mn, fluorescent groups such as FITC, rhodamine, Alexa, and quantum dots), and/or other moieties (e.g., ligands, antibodies, and/or portions thereof).

Antibodies as Targeting Agents

In some instances, the targeting agents are antigen binding proteins or antibodies or binding portions thereof. Antibodies can be generated to allow for the specific targeting of antigens or immunogens (e.g., tumor, tissue, or pathogen specific antigens) on various biological targets (e.g., pathogens, tumor cells, normal tissue). Such antibodies include, but are not limited to, polyclonal antibodies; monoclonal antibodies or antigen binding fragments thereof; modified antibodies such as chimeric antibodies, reshaped antibodies, humanized antibodies, or fragments thereof (e.g., Fv, Fab′, Fab, F(ab′)₂); or biosynthetic antibodies, e.g., single chain antibodies, single domain antibodies (DAB), Fvs, or single chain Fvs (scFv).

In certain instances, the targeting agent is an antibody the specifically binds an angiogenesis agent described herein. For example, the targeting agent is an anti-VEGF antibody described herein, e.g., bevacizumab. In other examples, the liposome or micelle includes, in addition to an anti-angiogenesis agent described herein, an additional antibody that targets additional ligands.

Methods of making and using polyclonal and monoclonal antibodies are well known in the art, e.g., in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)₂ fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1st edition).

Antibody attachment can be performed via any method that does not compromise the ability of the antibody to target a tumor, and to treat it, e.g., by binding to a specific anti-angiogenesis factor. For example, attachment can be performed through standard covalent binding to free amine groups (see, e.g., Torchilin et al., Hybridoma 6:229-240 (1987); Torchilin et al, Biochim. Biophys. Acta 1511:397-411 (2001); Masuko et al., Biomacromol. 6:800-884 (2005)).

Signal Peptides as Targeting Agents

In some instances, the targeting agents include a signal peptide. These peptides can be chemically synthesized or cloned, expressed and purified using known techniques. Signal peptides can be used to target the liposomes or micelles described herein to a discrete region within a brain cell.

Nucleic Acids as Targeting Agents

In other instances, the targeting agent is a nucleic acid (e.g., RNA or DNA). In some examples, the nucleic acid targeting agents are designed to hybridize by base pairing to a particular nucleic acid (e.g., chromosomal DNA, mRNA, or ribosomal RNA). In other situations, the nucleic acids bind a ligand or biological target. For example, the nucleic acid can bind reverse transcriptase, Rev or Tat proteins of HIV (Tuerk et al., Gene 137:33-9 (1993)); human nerve growth factor (Binkley et al., Nuc. Acids Res. 23:3198-205 (1995)); or vascular endothelial growth factor (Jellinek et al., Biochem. 83:10450-10456 (1994)). Nucleic acids that bind ligands can be identified by known methods, such as the SELEX procedure (see, e.g., U.S. Pat. Nos. 5,475,096; 5,270,163; and 5,475,096; and WO 97/38134; WO 98/33941; and WO 99/07724). The targeting agents can also be aptamers that bind to particular sequences.

Other Targeting Agents

The targeting agents can recognize a variety of epitopes on preselected biological targets (e.g., pathogens, tumor cells, or normal cells). For example, in some instances, the targeting agent can be sialic acid to target HIV (Wies et al., Nature 333:426 (1988)), influenza (White et al., Cell 56:725 (1989)), Chlamydia (Infect. Immunol. 57:2378 (1989)), Neisseria meningitidis, Streptococcus suis, Salmonella, mumps, newcastle, reovirus, Sendai virus, and myxovirus; and 9-OAC sialic acid to target coronavirus, encephalomyelitis virus, and rotavirus; non-sialic acid glycoproteins to target cytomegalovirus (Virology 176:337 (1990)) and measles virus (Virology 172:386 (1989)); CD4 (Khatzman et al., Nature 312:763 (1985)), vasoactive intestinal peptide (Sacerdote et al., J. of Neurosci. Research 18:102 (1987)), and peptide T (Ruff et al., FEBS Letters 211:17 (1987)) to target HIV; epidermal growth factor to target vaccinia (Epstein et al., Nature 318:663 (1985)); acetylcholine receptor to target rabies (Lentz et al., Science 215:182 (1982)); Cd3 complement receptor to target Epstein-Barr virus (Carel et al., J. Biol. Chem. 265:12293 (1990)); beta-adrenergic receptor to target reovirus (Co et al., Proc. Natl. Acad. Sci. USA 82:1494 (1985)); ICAM-1 (Marlin et al., Nature 344:70 (1990)), N-CAM, and myelin-associated glycoprotein MAb (Shephey et al., Proc. Natl. Acad. Sci. USA 85:7743 (1988)) to target rhinovirus; polio virus receptor to target polio virus (Mendelsohn et al., Cell 56:855 (1989)); fibroblast growth factor receptor to target herpes virus (Kaner et al., Science 248:1410 (1990)); oligomannose to target Escherichia coli; and ganglioside G_{M1} to target Neisseria meningitides.

In other instances, the targeting agent targets nanoparticles to factors expressed by oncogenes. These can include, but are not limited to, tyrosine kinases (membrane-associated and cytoplasmic forms), such as members of the Src family; serine/threonine kinases, such as Mos; growth factor and receptors, such as platelet derived growth factor (PDDG), small GTPases (G proteins), including the ras family, cyclin-dependent protein kinases (cdk), members of the myc family members, including c-myc, N-myc, and L-myc, and bcl-2 family members.

In addition, vitamins (both fat soluble and non-fat soluble vitamins) can be used as targeting agents to target biological targets (e.g., cells) that have receptors for, or otherwise take up, vitamins. For example, fat soluble vitamins (such as vitamin D and its analogs, vitamin E, vitamin A), and water soluble vitamins (such as vitamin C) can be used as targeting agents.

### Diseases/Disorders

The methods described herein can inhibit the growth, progression, and/or metastasis of hyperproliferative, hyperplastic, metaplastic, dysplastic, and pre-neoplastic diseases or disorders.

By “hyperproliferative disease or disorder” is meant a neoplastic cell growth or proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and cancer. Additional nonlimiting examples of hyperproliferative diseases, disorders, and/or conditions include neoplasms, whether benign or malignant, located in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

As used herein, the term “tumor” or “tumor tissue” refers to an abnormal mass of tissue that results from excessive cell division. A tumor or tumor tissue comprises “tumor cells”, which are neoplastic cells with abnormal growth properties and no useful bodily function. Tumors, tumor tissue, and tumor cells may be benign or malignant. A tumor or tumor tissue can also comprise “tumor-associated non-tumor cells”, such as vascular cells that form blood vessels to supply the tumor or tumor tissue. Non-tumor cells can be induced to replicate and develop by tumor cells, for example, induced to undergo angiogenesis within or surrounding a tumor or tumor tissue.

As used herein, the term “malignancy” refers to a non-benign tumor or a cancer. As used herein, the term “cancer” means a type of hyperproliferative disease that includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include squamous cell cancer (e.g., epithelial squamous cell cancer), lung cancer (including small-cell lung cancer, non-small cell lung cancer, adenocarcinoma of the lung and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer, uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to, Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Fibrosarcoma, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, and Wilm's Tumor.

The methods described herein can also be used to treat premalignant conditions and to prevent progression to a neoplastic or malignant state including, but not limited to, those disorders described above. Such uses are indicated in conditions known or suspected of preceding progression to neoplasia or cancer, in particular where non-neoplastic cell growth consisting of hyperplasia, metaplasia, or dysplasia has occurred (see, e.g., Robbins and Angell, Basic Pathology, 2d Ed., W.B. Saunders Co., Philadelphia, pp. 68-79 (1976)).

The methods described herein can further be used to treat hyperplastic disorders. Hyperplasia is a form of controlled cell proliferation, involving an increase in cell number in a tissue or organ, without significant alteration in structure or function. Hyperplastic disorders include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

The methods described herein can also be used to treat metaplastic disorders. Metaplasia is a form of controlled cell growth in which one type of adult or fully differentiated cell substitutes for another type of adult cell. Metaplastic disorders include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

The methods described herein can also be used to treat dysplastic disorders. Dysplasia can be a forerunner of cancer and is found mainly in the epithelia. Dysplasia is a disorderly form of non-neoplastic cell growth, involving a loss in individual cell uniformity and in the architectural orientation of cells. Dysplastic cells can have abnormally large, deeply stained nuclei, and exhibit pleomorphism. Dysplasia can occur, e.g., in areas of chronic irritation or inflammation. Dysplastic disorders include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of the jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders that can be treated by the methods described herein include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

### Therapeutic Administration

The route and/or mode of administration of a liposome or micelle described herein can vary depending upon the desired results. One with skill in the art, i.e., a physician, is aware that dosage regimens can be adjusted to provide the desired response, e.g., a therapeutic response.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. The mode of administration is left to the discretion of the practitioner.

In some instances, a liposome or micelle described herein (e.g., a pharmaceutical formulation of a liposome or a micelle) can effectively cross the blood brain barrier and enter the brain. In other instances, a liposome or micelle can be delivered using techniques designed to permit or to enhance the ability of the formulation to cross the blood-brain barrier. Such techniques are known in the art (e.g., WO 89/10134; Cloughesy et al., J. Neurooncol. 26:125-132 (1995); and Begley, J. Pharm. Pharmacol. 48:136-146 (1996)). Components of a formulation can also be modified (e.g., chemically) using methods known in the art to facilitate their entry into the CNS.

For example, in some instances, a liposome or micelle described herein is administered locally. This is achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers. In some situations, a liposome or micelle described herein is introduced into the central nervous system, circulatory system or gastrointestinal tract by any suitable route, including intraventricular, intrathecal injection, paraspinal injection, epidural injection, enema, and by injection adjacent to a peripheral nerve.

Pulmonary administration can also be employed, e.g., by use of an inhaler or nebulizer, and formulation with an aerosolizing agent, or via perfusion in a fluorocarbon or synthetic pulmonary surfactant.

A liposome or micelle described herein can be formulated as a pharmaceutical composition that includes a suitable amount of a physiologically acceptable excipient (see, e.g., Remington's Pharmaceutical Sciences pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one situation, the physiologically acceptable excipients are sterile when administered to an animal. The physiologically acceptable excipient should be stable under the conditions of manufacture and storage and should be preserved against the contaminating action of microorganisms. Water is a particularly useful excipient when a liposome or micelle described herein is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable physiologically acceptable excipients are described in Remington's Pharmaceutical Sciences pp. 1447-1676 (Alfonso R. Gennaro, ed., 19th ed. 1995). The pharmaceutical compositions, if desired, can also contain minor amounts of wetting or emulsifying agents, or pH buffering agents.

Liquid carriers can be used in preparing solutions, suspensions, emulsions, syrups, and elixirs. A liposome or micelle described herein can be suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carrier for pressurized compositions can be halogenated hydrocarbon or other pharmaceutically acceptable propellant.

In other instances, a liposome or micelle described herein is formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where a liposome or micelle described herein is administered by infusion, it can be dispensed, for example, with an infusion bottle containing sterile pharmaceutical grade water or saline. Where a liposome or micelle described herein is administered by injection, an ampule of sterile water for injection or saline can be provided so that the ingredients can be mixed prior to administration.

A liposome or micelle described herein can be administered rectally or vaginally in the form of a conventional suppository. Suppository formulations can be made using methods known to those in the art from traditional materials, including cocoa butter, with or without the addition of waxes to alter the suppository's melting point, and glycerin. Water-soluble suppository bases, such as polyethylene glycols of various molecular weights, can also be used.

The amount of a liposome or micelle described herein that is effective for treating disorder or disease can be determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. For example, the dose of a liposome or micelle described herein can each range from about 0.001 mg/kg to about 250 mg/kg of body weight per day, from about 1 mg/kg to about 250 mg/kg body weight per day, from about 1 mg/kg to about 50 mg/kg body weight per day, or from about 1 mg/kg to about 20 mg/kg of body weight per day. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy can be determined according to the judgment of a health-care practitioner.

In some instances, a pharmaceutical composition described herein is in unit dosage form, e.g., as a tablet, capsule, powder, solution, suspension, emulsion, granule, or suppository. In such form, the pharmaceutical composition can be sub-divided into unit doses containing appropriate quantities of a nanoparticle described herein. The unit dosage form can be a packaged pharmaceutical composition, for example, packeted powders, vials, ampoules, pre-filled syringes or sachets containing liquids. The unit dosage form can be, for example, a capsule or tablet itself, or it can be the appropriate number of any such compositions in package form. Such unit dosage form can contain from about 1 mg/kg to about 250 mg/kg, and can be given in a single dose or in two or more divided doses.

### Kits

A liposome or micelle described herein can be provided in a kit. In some instances, the kit includes (a) a container that contains a liposome or micelle and, optionally (b) informational material. The informational material can be descriptive, instructional, marketing or other material that relates to the methods described herein and/or the use of the liposome or micelle, e.g., for therapeutic benefit.

The informational material of the kits is not limited in its form. In some instances, the informational material can include information about production of the liposome or micelle, molecular weight of the liposome or micelle, concentration, date of expiration, batch or production site information, and so forth. In other situations, the informational material relates to methods of administering the liposome or micelle, e.g., in a suitable amount, manner, or mode of administration (e.g., a dose, dosage form, or mode of administration described herein). The method can be a method of treating a subject having a disorder.

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about the nanoparticles therein and/or their use in the methods described herein. The informational material can also be provided in any combination of formats.

In addition to the liposome or micelle, the kit can include other ingredients, such as a solvent or buffer, a stabilizer, or a preservative. The kit can also include other agents, e.g., a second or third agent, e.g., other therapeutic agents. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for the liposomes or micelles or other agents. In some cases, the kit contains separate containers, dividers or compartments for the liposomes or micelles and informational material. For example, the liposomes or micelles can be contained in a bottle, vial, or syringe, and the informational material can be contained in a plastic sleeve or packet. In other situations, the separate elements of the kit are contained within a single, undivided container. For example, the liposomes or micelles can be contained in a bottle, vial or syringe that has attached thereto the informational material in the form of a label. In some cases, the kit can include a plurality (e.g., a pack) of individual containers, each containing one or more unit dosage forms (e.g., a dosage form described herein) of the liposomes or micelles. The containers can include a unit dosage, e.g., a unit that includes the liposomes or micelles. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of the liposomes or micelles, e.g., a syringe or other suitable delivery device. The device can be provided pre-loaded with liposomes or micelles, e.g., in a unit dose, or can be empty, but suitable for loading.

The invention is further illustrated by the following examples. The examples are provided for illustrative purposes only. They are not to be construed as limiting the scope or content of the invention in any way.

### EXAMPLES

### Example 1

### Conjugation of Bevacizumab to Cationic Liposomes Enhances Tumor Targeting

### A. Materials

DOTAP, cholesterol, DOPC, DOPE-PEG₂₀₀₀, DSPE-PEG₂₀₀₀-biotin, DOPE-FITC and DMPE-DTPA were obtained from Avanti Polar Lipids (Alabaster, Ala.). Phosphate-buffered saline (PBS) without calcium or magnesium was obtained from Biowhittaker (Nkialkersville, Md.) and fetal bovine serum (FMS) Fetalclone I optimized for hybridomas was obtained from Hyclone (Logan, Utah). Capan-1, PANC-1 and HPAF-II, pancreatic cell lines, and MS1-VEGF, murine pancreatic endothelial cells transfected to secrete VEGF, were obtained from American Type Cell Collections (ATCC, Manassas, Va.). Capan-1 was maintained in Iscove's modified Dulbecco's medium. PANC-1 and MS1-VEGF were cultured using Dulbecco's modified Eagle's medium. HPAF-II was grown using Eagle's minimum essential medium. All media was obtained from ATCC. Each growth media was supplemented with FBS 10%. HMEC-1 cells were obtained from the Centers for Disease Control and Prevention (Atlanta, Ga.). HMEC-1 cells were grown in either endothelial basel medium (EBM) with microvascular endothelial growth medium (EGM-MV) growth factors, supplemented with FBS 10%, or ERM-2 with EGM-2 MV, which contains FBS 5% from Lonza (Basel, Switzerland). HMEC-1 cells grown in EBM-2 with EGM-2 MV contain 2 ng/ml of VEGF 165. All cell lines were grown in a Revco ELITE III cell culture incubator (Kendro Laboratory, Asheville, N.C.) with 5% CO₂ at 37° C.

### B. Preparation of Liposomes

Required amounts of DOPC, cholesterol, DOPE-PEG₂₀₀₀, DSPE-PEG₂₀₀₀-biotin and DOTAP were mixed in appropriate ratios to prepare cationic and nominally electroneutral liposomes. Cholesterol, DOPE-PEG₂₀₀₀, and DSPE-PEG₂₀₀₀-Biotin content remained fixed in all preparations at 10, 5 and 0.2 mol %, respectively. For cationic liposomes DOTAP was added at 50 mot % with 35 mot % DOPC and for nominally electroneutral liposomes DOPC was maintained at 85 mol %. Typically, 1-2 mol % DOPE-FITC was included as part of the liposome preparation for studies involving fluorescence detection. Concentration of lipid used to prepare liposomes depended on the specific experiment and was typically between 10 and 20 μmol/ml. Chloroform was used to prepare lipid stocks and was evaporated after appropriate ratios were prepared using a Buchi Rotovapor R-200 (Buchi Labortechnik AG, Flawil, Switzerland) for 20 min, or until a dried thin lipid film was formed. Additional trace amounts of organic solvent were removed from the film by drying for 2 h in a vacuum environment using a Labconco freeze dryer (Labconco corporation, Kansas City, Mo.). The film was next hydrated with warm PBS in an inert atmosphere, incubated at 37° C. for at least 1 h, and vortexed intermittently. To reduce liposome size, liposomes were sonicated in a bath-type sonicator (Laboratory Supplies Corporation, Hicksville, N.Y.) for 10 min. This caused the large multilamellar heterogeneous vesicles to become a homogeneous population of small unilamellar vesicles. The liposomes were then filtered through a 0.22-μm filter. Following the completion of all steps, the liposome size (using dynamic light scattering principles and Stokes-Einstein equation and zeta-potential was measured at 25° C. in distilled water using a 90PLUS particle size and zeta-potential analyzer (Brookhaven Instruments, NY, USA). Each value represents the mean±standard deviation of four values for the nominally electroneutral Liposomes, and five values for the cationic liposomes for both the modified and unmodified varieties.

To prepare bevacizumab-modified cationic or nominally electroneutral liposomes the film was hydrated in a volume to account for the additional volume needed to add neutravidin (Pierce, Rockford, Ill., USA) and biotin-modified bevacizumab prepared following procedures provided by either Invitrogen or Pierce using biotin with 30.5 Å long spacer) and then sonicated (step 1). With hydrated liposomes, a 1:1 mot ratio of neutravidin to DSPE-PEG(2000)-biotin was added to the liposomes and incubated at room temperature for approximately 75 min (step 2). Unbound neutravidin was removed through dialysis with PBS overnight at 4″C using a 300 MWCO membrane with at least two changes of the dialysis PBS (step 3), Biotin-labeled bevacizumab was then added to the liposomes in a ratio of 1 mg of antibody per 10 of liposomes and incubated at room temperature for approximately 75 min (step 4). Unbound antibody was removed through dialysis with PBS overnight at 4° C. using a 300 K MWCO membrane with at least two changes of the dialysis PBS (step 5). The liposomes were then filtered through a 0.22-μm filter (step 6). For unmodified liposomes, an equal volume of PBS was added instead of neutravidin or biotin-modified bevacizumab.

### C. Bevacizumab/Liposome Cell Toxicity Assay

Cells were seeded at 1×10⁴ cells/well in 1 ml volumes in a 48-well plate. The cells were incubated for 24 h in a 37° C. cell culture incubator set at CO₂ 5%. Bevacizumab/liposomes were then added to each well at various concentrations. After 24 h of incubation the SRB assay was performed (as described in, e.g., Dandamudi et al., Biochim. Biophys. Acta 1768:427-438 (2007); Skehan et al., J. Natl. Cancer Inst. 82:1107-1112 (1990); and Kalra et al., Pharm. Res. 23:2809-2817 (2006)). Briefly, the plates were washed twice with 1 ml/well of 1×PBS. Then, 100 of trichloracetic acid 50% solution (TCA; 100% w/v) was added to each well and the plates were stored at 4° C. for 1 h. The plates were thoroughly washed with distilled H₂O five times and then 200 μl of SRB 0.4% was added to each well The plates were stored at room temperature for 30 min and then washed 4-6 times thoroughly with acetic acid 1% to remove excess SRB and air dried. 1 ml of PBS was added to each well to solubilize the SRB and fluorescence intensity was measured at excitation wavelength of 540/20 and emission wavelength of 590/20 nm using FLX 800 Fluorescence Microplate Reader (Biotech instruments Inc., Winooski, Vt.). All groups were compared with untreated cells for statistical analysis. The bevacizumab toxicity study was performed between three and six replicates depending on the cell line, and the liposome toxicity study was performed in triplicate.

### D. Cell-Liposome Interactions

Cells were seeded at 1×10⁴ cells/well in 1 ml volumes into 48-well plates and incubated at 37° C. with CO₂ 5% for 24 h. Fluoroscein-labeled liposomes were then added to the existing media and incubated for an additional 24 h. The plates were then washed with 1 ml/well of PBS twice. The fluorescence intensity was measured before and after washing the plate with a FLX800 Microplate Fluorescence Reader (Biotek Instruments Inc., Winooski, Vt.). Fluorescence intensity was measured to determine percent of liposomes associated with cells using a fixed excitation and emission wavelengths set at 485/20 and 530/20, respectively. Percent cell association was determined using the following formula: fluorescence intensity (arbitrary units) after washing/fluorescence intensity (arbitrary units) before washing×100. T-tests were used to evaluate statistically significant differences between experimental groups. Level of statistical significance reported for tests were set at *p≦0.05, **p≦0.01 and ***p≦0.001. The replicates for each group were between three and six depending on the cell line employed.

### E. Fluorescence/DIC Microscopic Analysis

Fluorescence microscopy was used to evaluate cellular uptake of liposomes in human and murine pancreatic and endothelial cells. Fluoroscein (DSPE-FITC) at 1 mol % was used to prepare liposomes to track areas of localization within cells by fluorescence microscopy. The cells were harvested from flasks using trypsin-EDTA and subsequently seeded at 5×10⁵ cells per well in 1 ml of media on cover slips (22 mm square No. 1½ coming glass made from No, 0211 zinc titania from Corning, Lowell, Mass.) in six-well plates for a period of 24 h in a humidified atmosphere of CO₂ 5% at 37° C. Liposome preparations were added (100 nmoles) to the existing medium and the plate was incubated for an additional 6 h. Next, the cover slip was washed with PBS to remove all unbound liposomes and cellular debris. Cover slips were next mounted on slides using slowFade Gold antifade reagent with DAPI (Invitrogen Inc, Eugene, Oreg.), and analyzed using DIC and fluorescence microscopy with a BX61 WI Olympus fluorescence microscope from Optical Analysis Corporation (Melville, N.Y.). Images were acquired at 20× magnifications and recorded with an intensified CCD camera. The DIC and fluorescent images were acquired in duplicate.

### F. Liposomal FACS Analysis

Cells were seeded at 5×10⁵ cells/well in 1 ml of media in a six-well plate in their appropriate media and growth supplements and incubated overnight in a CO₂ 5% and 37° C. incubator, FITC-labeled liposomes (100 nmoles) were then added to the wells. The plate was incubated for 1 h in a CO₂ 5% and 37° C. incubator, washed twice with PBS, trypsinized and washed once with PBS. The cells were then analyzed with a FACScaliber (BD Biosciences, San Jose, Calif.). The FACS analysis was performed in duplicate.

### G. VEGF ELISA

Cells were seeded at 1×10⁴ cells per well in 1 ml of media. In total, 600 μl of media was removed from each well at the appropriate times following the seeding of the plate (24, 48, 72, 96, 120 and 144 h) and placed into microcentrifuge tubes. The same plate was then assessed for cell growth for each time point using the SRB assay. The media was not changed during any of the time points. The supernatant collected was then centrifuged at 1000 g for 5 min to remove any debris from the supernatant and then frozen at −80° C. until assayed for VEGF ELISA was performed following instructions provided by manufacturer of ELISA kit (Pierce, Rockford, Ill.) to determine the levels of VEGF in the supernatant. The amount of VEGF released was tested in duplicate. The growth of each cell type over the six time points was evaluated with six replicates.

### H. Biodistribution

Biodistribution studies were executed using female SCID mice (2-4 mice per group). Capan-1 cells (3×10⁶ cells) were injected subcutaneously into the right hind flank of all the mice between 8 and 10 weeks old. The tumors were allowed to grow to approximately 100 mm³ before injection (about 22 days from day of tumor cell implantation). Bevacizumab-modified (and unmodified) PCLs were prepared with 1 mot % DMPE-DTPA to chelate ¹¹¹In to the liposome for detection. Modified liposomes had a mole ratio of bevacizumab:DSPE-PEG₂₀₀₀-biotin. Approximately 500 nmoles (100-200 μl) of radiolabeled liposomes were injected via tail vein. Mice were anesthetized with isoflurane approximately 24 h postadministration of formulations. Collection of blood was done through the retro-orbital sinus and then sacrificed by cervical dislocation. Muscle, lung, liver, spleen, kidney and tumor were removed, weighed, and assessed for the corresponding levels of radioactivity recovered using a Beckman Gamma 550 B counter (Fullerton, Calif.). The biodistribution was assessed as percent of injected dose per gram of tissue as per Equation 1 (CPM—counts per min).

$$\frac{\begin{matrix}CPM values in each organ \text{/} weight \\ of each organ in grams\end{matrix} }{CPM values of injected dose} \times 100 \qquad ( 1 )$$

### I. Statistical Analysis

T-tests were used to evaluate statistically significant differences between experimental groups. The normality of the data was not performed following the completion of the t-test. Level of statistical significance reported for tests were set at *p≦0.05, **p≦0.01 and ***p≦0.001.

### J. Results

1. VEGF Production and Secretion by Cell Lines

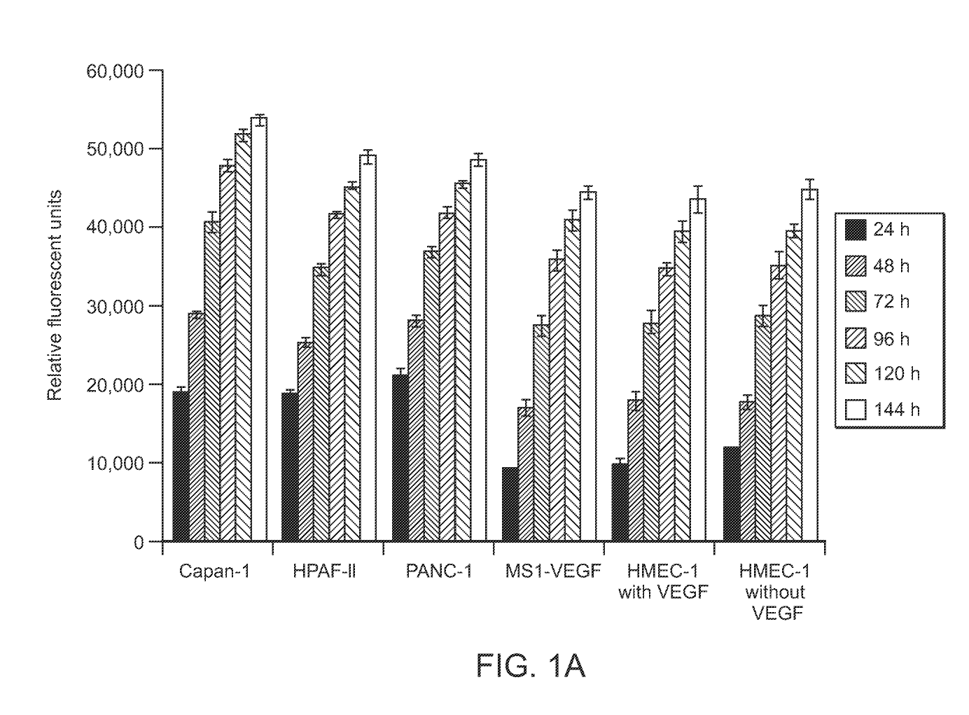

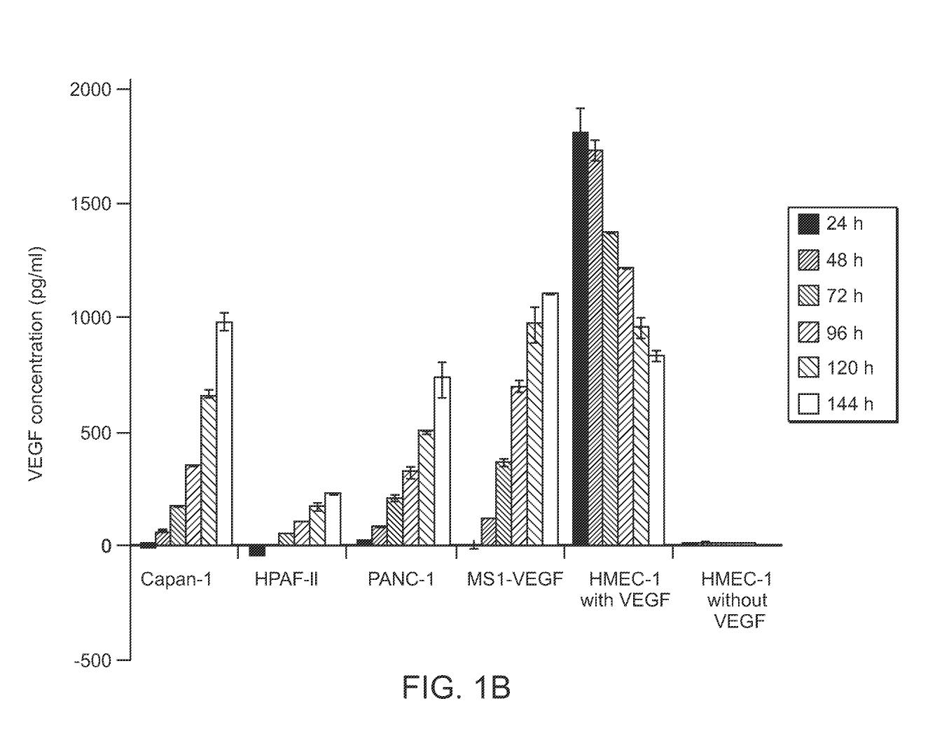

To determine whether the modification of cationic liposomes with bevacizumab would potentially increase vascular and tumor targeting, the ability of the cell lines to produce (and secrete) VEGF was assessed over a period of 6 days. All six cell lines continued to grow over the 6 days, and without exception, none of the cell lines ever reached confluency (FIG. 1A). Confluence was evaluated in parallel using inverted light microscopy. This suggested that a healthy, nutrient-rich environment was maintained throughout the experiment. All VEGF levels were evaluated at their respective time points with no external interference. Therefore, the VEGF levels represent the total from seeding until the time of sampling.

Capan-1, HPAF-II and PANC-1 are three pancreatic cancer cell lines that steadily produced and secreted VEGF over a period of 6 days (FIG. 1B). MS1-VEGF is an endothelial cell line that had been transformed to secrete VEGF (FIG. 1B). HMEC-1 cells are endothelial cells that were grown in media containing VEGF or media without VEGF. HMEC-1 cells grown in media containing VEGF did not produce or secrete VEGF, but the levels decreased steadily over the 6-day period. HMEC-1 cells grown in media without VEGF did not secrete VEGF into the growth medium, nor did they require the presence of the growth factor to grow (FIGS. 1A and 1B). The cells were capable of growing in the absence or presence of VEGF. When VEGF is present along with FBS 5% the cells use the VEGF to grow. When VEGF is not present, but contains FBS 10%, the cells can grow under these conditions as well.

2. Physicochemical Characterization of Bevacizumab-Modified Liposomes

The conjugation of bevacizumab to the distal end of PEG on cationic Liposomes was a multistep process. First, liposomes were prepared containing a small amount of DSPE-PEG₂₀₀₀-biotin. Once liposomes were formed and sonicated, neutravidin was coupled to biotin on the liposome surface. The ratio of biotin to neutravidin was 1:1, Next, the unbound neutravidin was removed through dialysis and biotinylated bevacizumab was added; the biotin was then bound to neutravidin. Dialysis was performed to remove unbound bevacizumab. The liposomes were finally filtered through a 0.22 μm filter prior to use. The modification of PCLs with bevacizumab did not result in a statistically significant change in zeta-potential, showing 35±4 MV and 31±4 MV for the unmodified and modified PCLs, respectively. No difference in zeta-potential was Observed between the modified (−29±6 mV) and unmodified (−32±10 mV) nominally electroneutral liposomes. However, the average liposome size diameters for the unmodified (control) nominally electroneutral and cationic liposomes remained unchanged throughout the liposome preparation process (Table 1; steps 1-6), but the addition of neutravidin (Table 1; step 2) resulted in a significant increase in liposome size. A general trend towards an additional increase in liposome size was observed following the addition of biotinylated bevacizumab (Table 1; step 4). For both the addition of neutravidin and biotinylated bevacizumab, the dialysis procedure (used for separation of bound reactant from unbound material) had no observable effect on liposome size (Table 1; steps 3 and 5). The overall increase in liposome size, when compared with the antibody-modified PCB, was probably due to the efficient conjugation of both neutravidin and the biotinylated antibody to the distal end of PEG on the liposome surface.

TABLE 1
Particle size and -potential of bevacizumab-modified and -unmodified
pegylated cationic liposomes, and bevacizumab-modified and unmodified
nominally electroneutral liposomes.

| | | | | | |
|---|---|---|---|---|---|
| Step 1 | Step 2 | Step 3 | Step 4 | Step 5 | Step 6 |

Particle size (nm)

| | | | | | | |
|---|---|---|---|---|---|---|
| Cationic SLs | 124 ± 5 | 123 ± 5 | 125 ± 5 | 124 ± 5 | 123 ± 10 | 125 ± 8 |
| Cationic ILS | 124 ± 5 | 166 ± 23 | 174 ± 24 | 207 ± 44 | 220 ± 44 | 212 ± 35 |
| Electroneutral SLs | 114 ± 18 | 114 ± 18 | 116 ± 17 | 118 ± 23 | 120 ± 20 | 119 ± 15 |
| Electroneutral ILs | 113 ± 18 | 156 ± 5 | 162 ± 11 | 189 ± 34 | 199 ± 33 | 185 ± 26 |

ζ-potential (mV)

| | | | | | | |
|---|---|---|---|---|---|---|
| Cationic SLs | 39 ± 11 | 40 ± 11 | 39 ± 11 | 35 ± 10 | 36 ± 6 | 35 ± 4 |
| Cationic ILS | 41 ± 10 | 33 ± 6 | 30 ± 6 | 28 ± 8 | 28 ± 7 | 31 ± 4 |
| Electroneutral SLs | −26 ± 3 | −32 ± 3 | −32 ± 4 | −28 ± 7 | −34 ± 4 | −32 ± 10 |
| Electroneutral ILs | −26 ± 3 | −26 ± 3 | −26 ± 6 | −26 ± 5 | −28 ± 9 | −29 ± 6 |

IL: Bevacizumab-modified liposome;
SL: Unmodified liposomes.

3. Influence of Bevacizumab on Cellular Toxicity of Liposomes

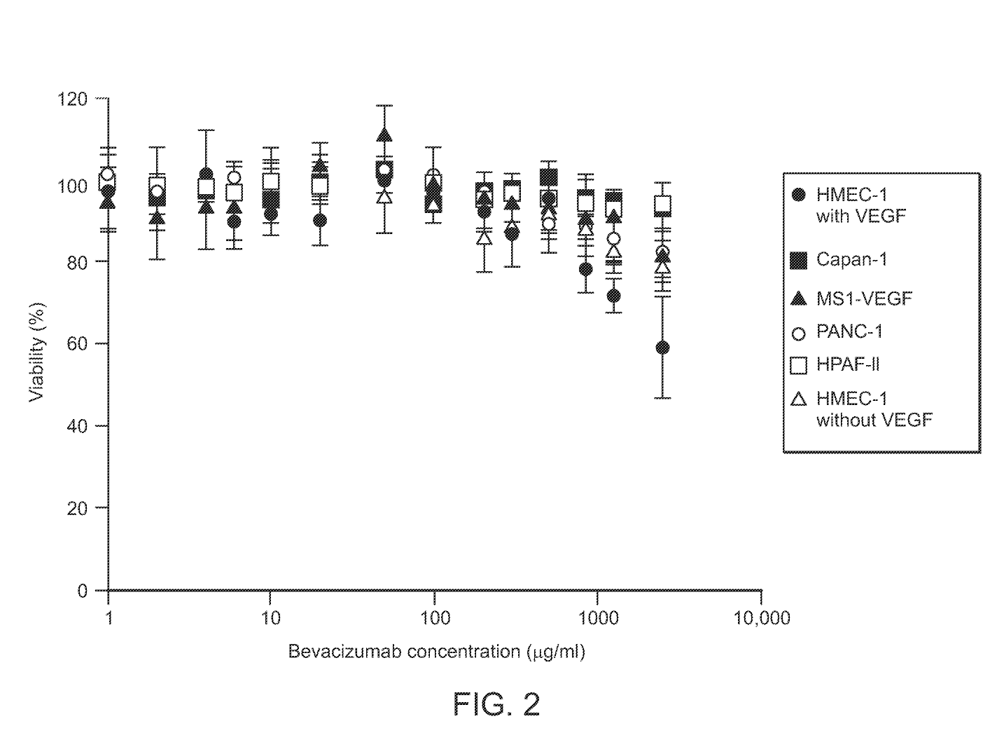

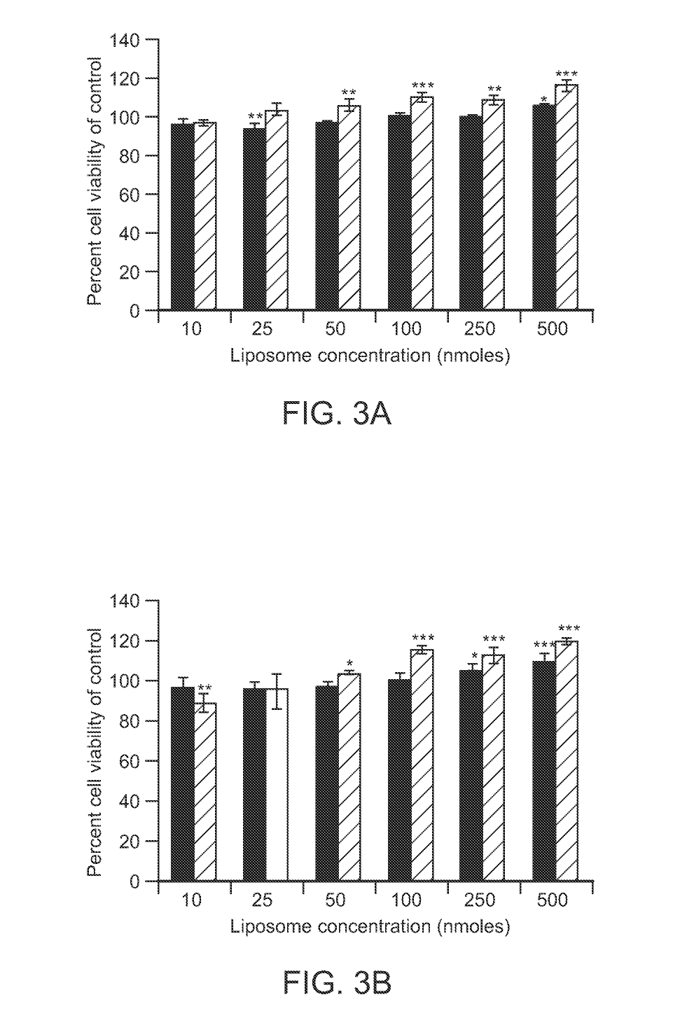

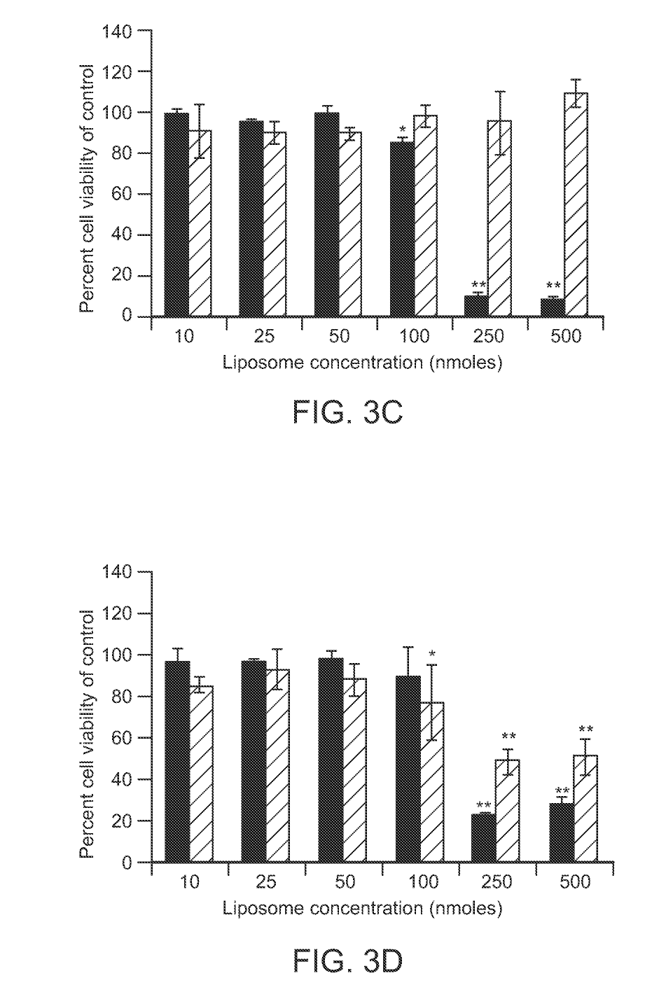

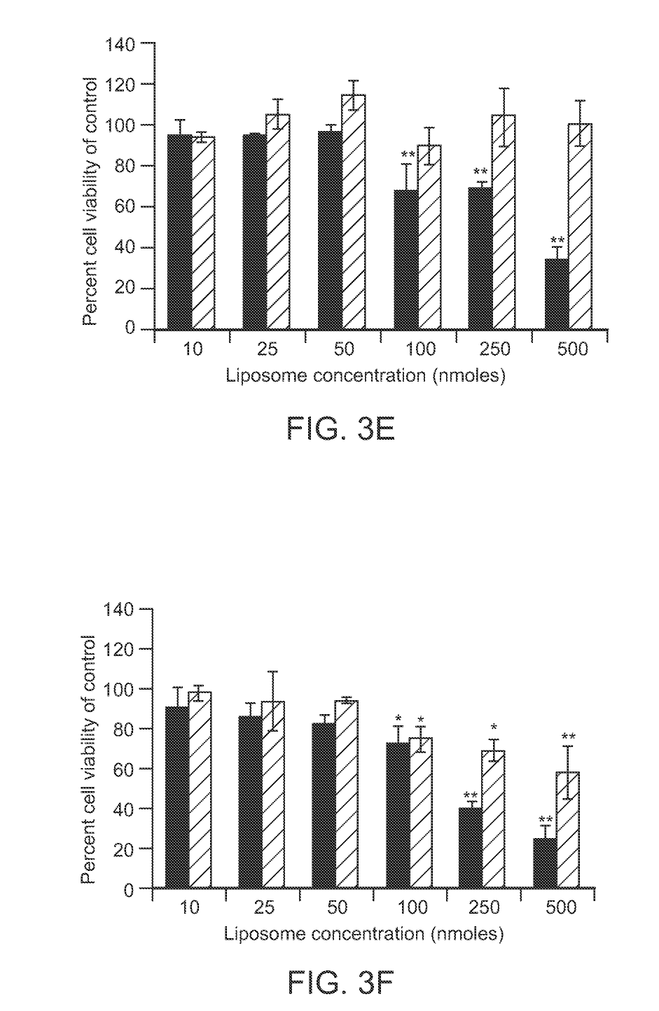

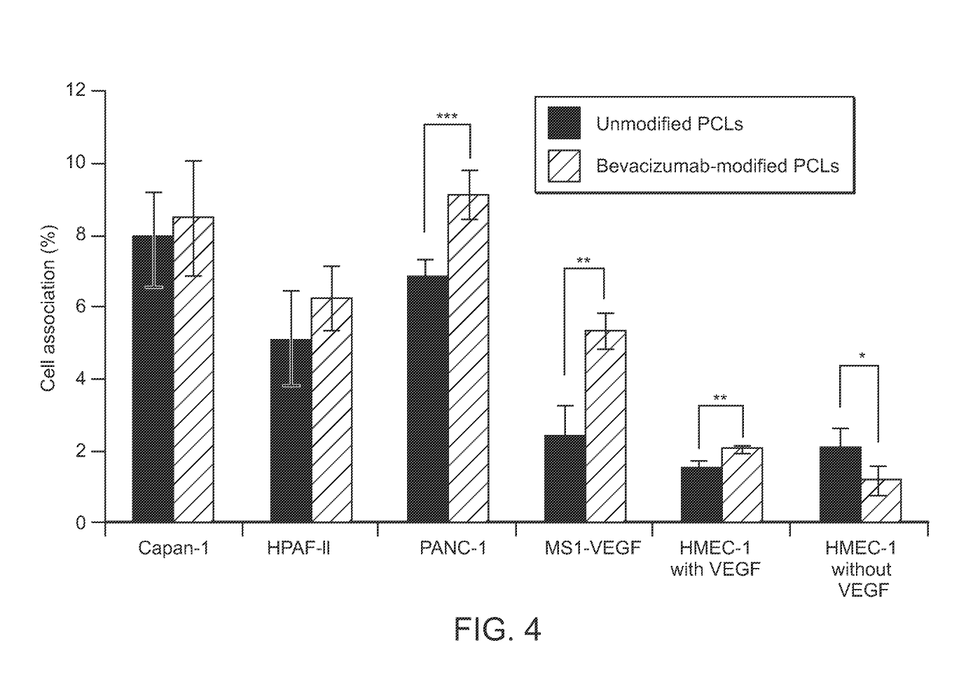

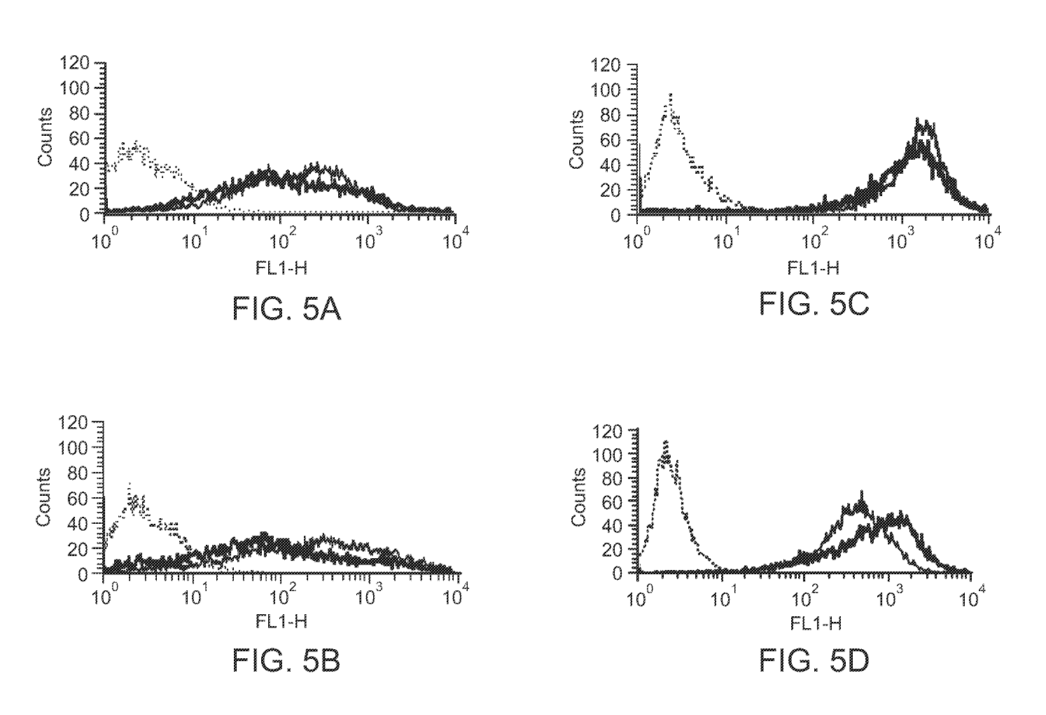

The nontoxic concentration of bevacizumab was determined in order to establish the maximum amount of antibody that could be used without altering the growth potential of the cells, which was determined to be 500 ng/ml (FIG. 2). To assess the ability of the unmodified and modified PCLs to associate with, and be taken up by cells, the maximum nontoxic concentration was determined for each cell line. For two of the pancreatic cancer cell lines, Capan-1 and HPAF-II, the nontoxic concentration was greater then 500 mmoles/ml for both formulations used. For PANC-1 and the endothelial cell (MS1-VEGF, HMEC-1 with and without VEGF in the media) less than or equal to 100 nmoles/ml of the unmodified preparation was relatively nontoxic to the cells. For PANC-1 and HMEC-1 cells grown in the presence of VEGF, the nontoxic concentration for bevacizumab-modified PCLs was 500 nmoles/ml or more. However, for MS1-VEGF and HMEC-1 cells grown without VEGF in the media, a concentration of 100 nmoles/ml or less of the bevacizumab-modified PCLs was relatively nontoxic to the cells. (FIG. 3). The data suggest that the modification provided some form of protection against the toxicity of PCLs in vitro. Interestingly, bevacizumab-modified PCLs appeared to support the growth potential of Capan-1 and HPAF-II cells compared with untreated control group (FIGS. 3A and 3B).

4. Effect of Bevacizumab PCL Uptake by Cells

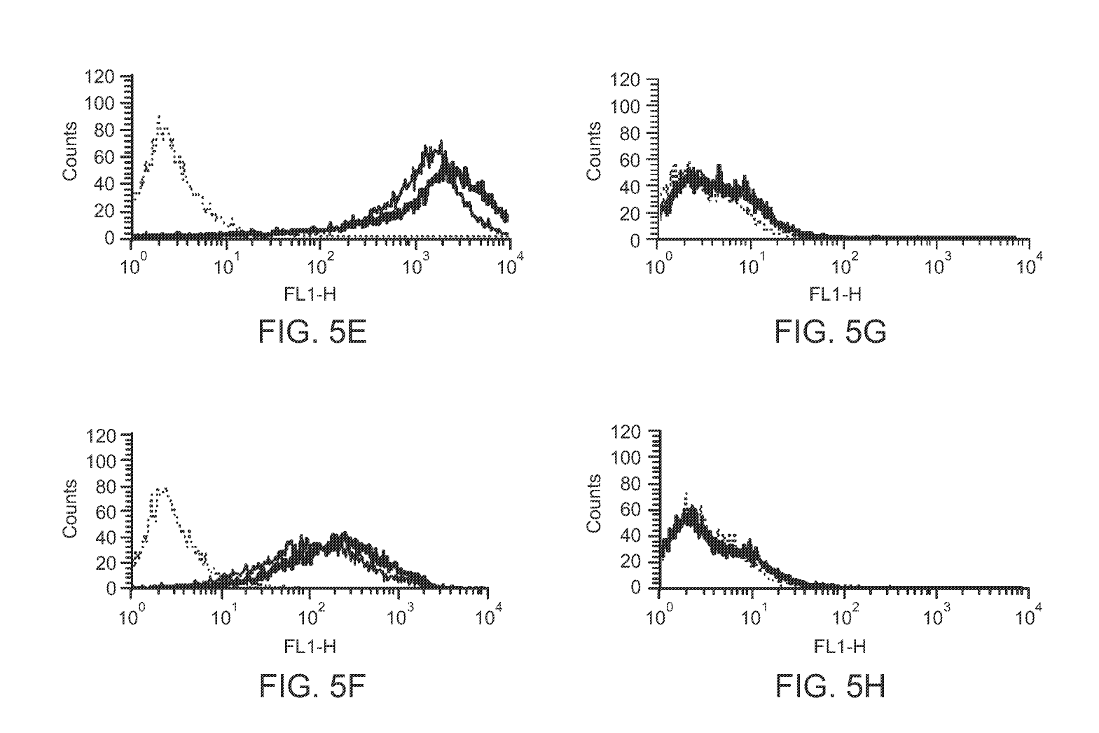

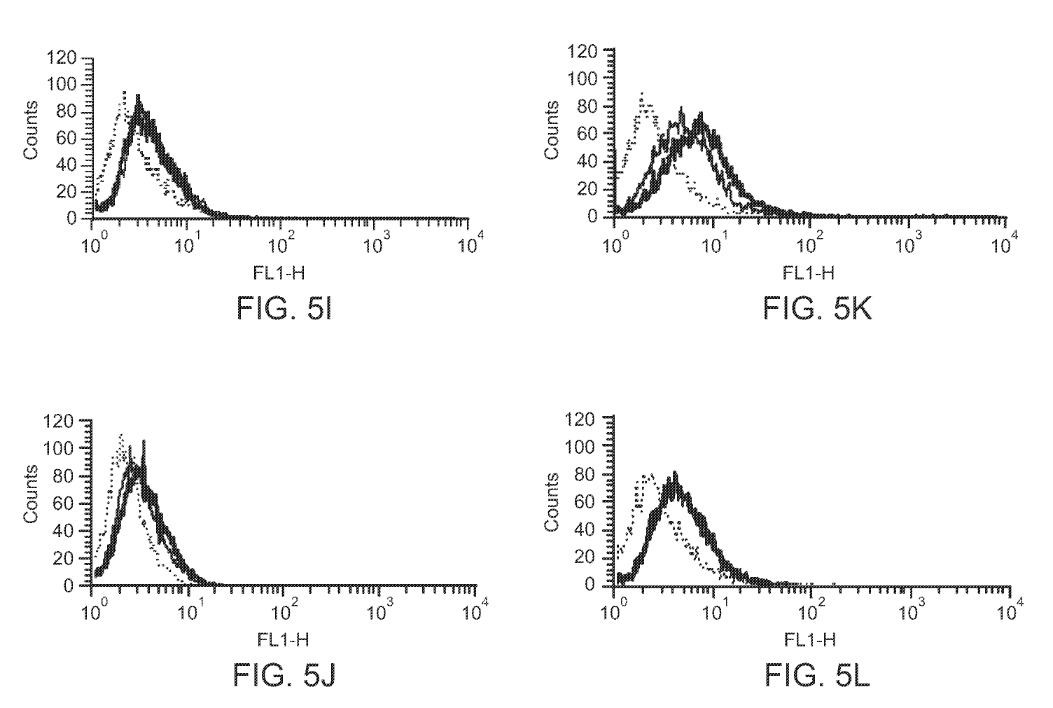

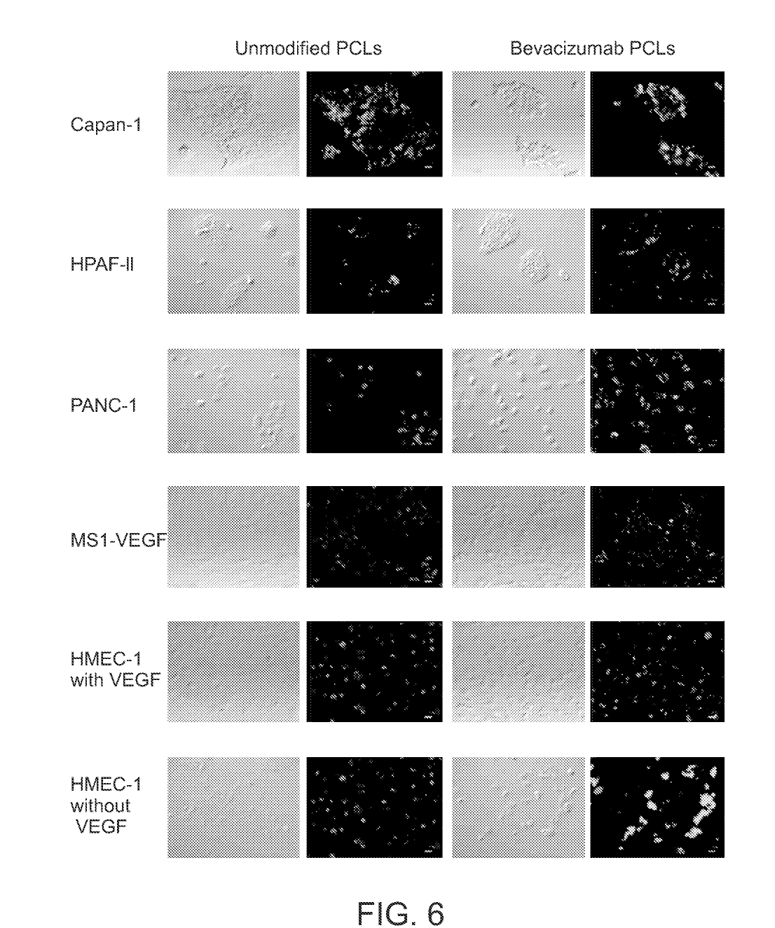

The conjugation of bevacizumab to the terminal ending of PEG of PCLs significantly increased cellular uptake of the liposomes by PANC-1, MS1-VEGF as well as HMEC-1 cells, when grown in the presence of VEGF (FIG. 4, *—p≦0.05). However, no difference was observed with Capan-1 and HPAF-II cells when bevacizumab was added to the surface of the liposomes (FIG. 4). In the case of HMEC-1 cells grown in medium without VEGF, there was a significant decrease in cellular uptake of bevacizumab-conjugated liposomes. A similar trend was observed with FACS studies, where more bevacizumab-conjugated liposomes were taken up by the MS1-VEGF and HMEC-1 cells grown in the presence of VEGF (FIGS. 5D and 5E). Capan-1, HPAF-II, PANC-1 and HMEC-1 cells grown in the absence of VEGF showed a similar degree of uptake when comparing bevacizumab and unmodified PCLs (FIGS. 5A-5C and 5F). The HMEC-1 cells grown in the presence of VEGF had taken up liposomes in general to a greater extent compared with HMEC-1 cells grown without VEGF. The uptake of nominally electroneutral modified or control (unmodified) PEGylated liposomes was significantly lower than the cationic variety (FIG. 5). Even though the significant increase in cellular uptake was not observed with all six cell lines through FACS and cell association studies, the fluorescent microscopic images suggested a greater degree of cellular uptake for the modified PCLs (FIG. 6). HMEC-1 cells grown without VEGF appeared different from the other cell lines. The liposomes covered the surface of the cells, representing that many of the liposomes were not taken up by the cell, but remained associated with the cell surface. All of the cell lines grown in the presence of VEGF (Capan-1, HPAF-II, PANC-1, MS1-VEGF and HMEC-1) appeared to demonstrate intracellular accumulation of the drug carrier molecule, particularly in areas near (but not within) the nuclear compartment of the cells (FIG. 6).

5. Organ and Tumor-specific Liposome Uptake

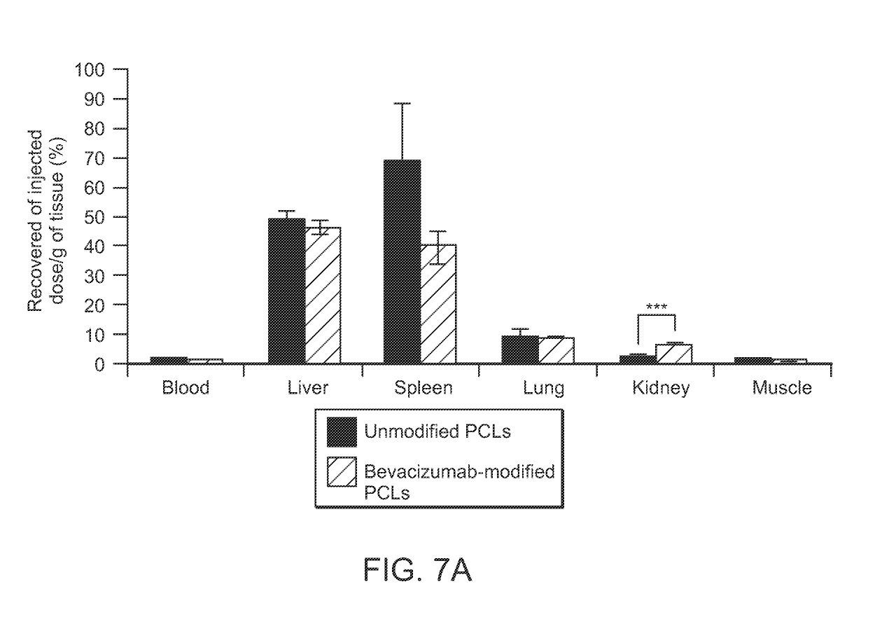

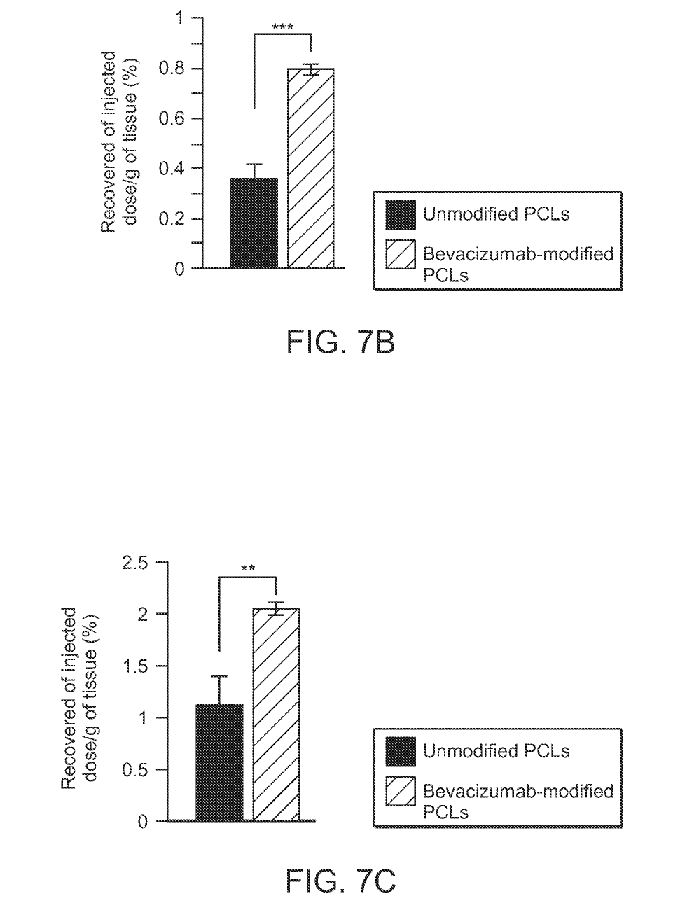

The coupling of bevacizumab to the surface of PCLs significantly increased the blood and kidney distribution, with decreased uptake by the spleen (FIGS. 7A and 7B). In general, the distribution of the modified and unmodified PCLs in the remaining organs was similar (FIG. 7C). The targeting potential of antibody-modified. PC Ls in the tumor was greater, given that the percentage of the injected dose recovered per gram of the tumor increased significantly (FIG. 7B). It is possible to improve tumor-specific uptake by increasing the liposome dose. The fraction of the injected dose recovered by the tumor increases at the expense of the other organs, given that the level of VEGF produced by tumors is significantly greater than in normal healthy tissues. Soluble VEGF produced by a developing tumor also binds to circulating bevacizumab-modified PCB to a significant extent compared with the unmodified variety, thus improving the tumor-targeting of PCLs.

### Example 2

### In Vivo Therapeutic Studies using Pancreatic Tumor Model

### A. Methods

Male nude mice, 7 to 8-weeks-old, were obtained from Charles River Laboratories (Wilminton, Mass.). Tumors were grown by subcutaneously injecting 2×10⁶ HPAF-II cells in 0.1 ml of cell culture media. Daily tumor volume measurements were taken using an electronic digital caliper (Control Company, Friendswood, Tex.). The equation a²×b×0.52 was used to calculate the tumor volume, where ‘a’ and ‘b’ corresponded to the longer and shorter diameters, respectively. Mice were divided into four groups, (1) untreated control, (2) 2-methoxyestradiol-loaded unmodified PEGylated cationic liposomes (“PCLs”), (3) bevacizumab alone, and (4) 2-methoxyestradiol-loaded bevacizumab-modified PCLs. Animals received injections of the various formulations when the tumors reached a size of about 150 mm³; the untreated control group received no injections. The other three groups had a dosing schedule of 4 injections, one injection every 3 days. Day 1 injection was 18 μg of 2-methoxyestradiol for both PCL formulations. Day 4, 7, and 10 injections corresponded to 24 μg of 2-methoxyestradiol for both formulations. The injections of bevacizumab alone corresponded to a similar amount of bevacizumab conjugated to the 2-methoxyestradiol-loaded bevacizumab-modified PCLs. On day 1, 300 μg of bevacizumab was injected and on days 4, 7, and 10, 400 μg of bevacizumab was injected intravenously into the tail vein of mice. The end of the study was three days after the fourth injection, at which point the mice were anesthetized with ketamine/xylazine. The mice were sacrificed, liver, lung, spleen and tumor tissue were removed surgically and fixed in 10% formalin at 4° C. for about 24 hours and then transferred to PBS pH 7.4 for histochemical staining and analysis.

A second study was done using male SCID mice obtained from Charles River Laboratories (Wilminton, Mass.) that were subcutaneously injected with 2×10⁶ Capan-1 cells in 0.1 mL of culture media. The tumors were allowed to grow to about 150 mm³ before initiation of the treatment. The mice were divided into three groups, (1) untreated, (2) 2-methoxyestradiol-loaded unmodified PCLs and (3) 2-methoxyestradiol-loaded bevacizumab-modified PCLs. The dosing schedule was the same as the HPAF-II efficacy studies except that on days 1, 4, 7, and 10, twice as much formulation was injected. However, the total amount of antibody used was the same as in the HPAF-II study (about 400 μg per injection). The end of the study was three days after the fourth injection, at which point the mice were anesthetized with ketamine/xylazine. The mice were sacrificed, liver, lung, spleen and tumor tissue were removed surgically and fixed in 10% formalin at 4° C. for about 24 hours, and then transferred to PBS (pH 7.4) for histochemical staining and analysis.

### B. Results

To evaluate the benefit of attaching bevacizumab to the distal end of PEG on PCLs, an in vivo subcutaneous HPAF-II pancreatic tumor model in nude mice was used. This model was used to determine if the benefits seen in vitro translated to in vivo treatment. 2-methoxyestradiol was chosen as the therapeutic drug because it is known to be anti-angiogenic as well as an anti-tumor agent.

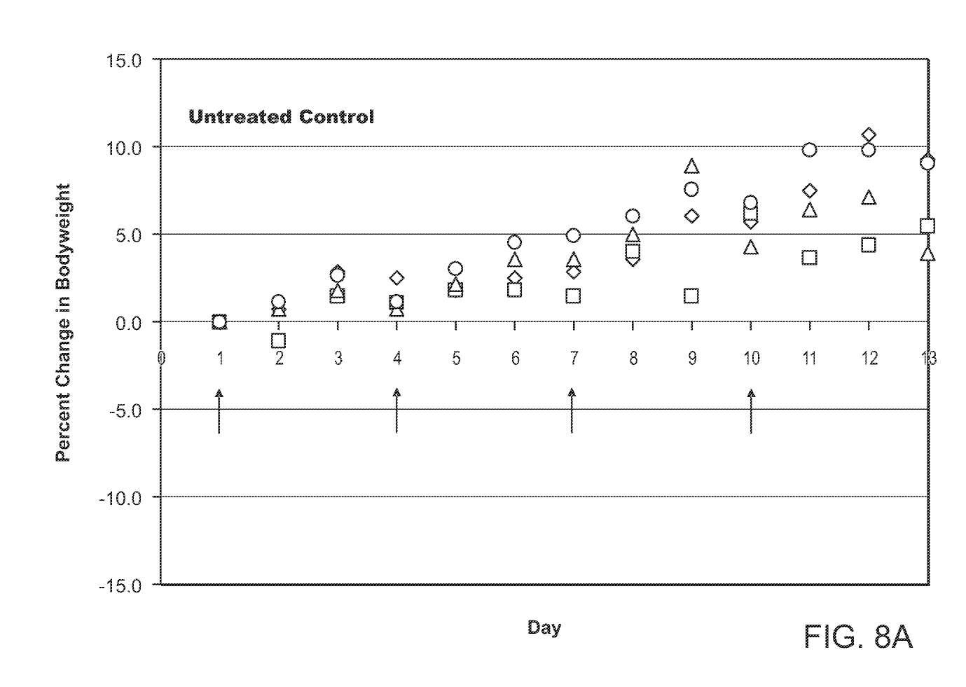

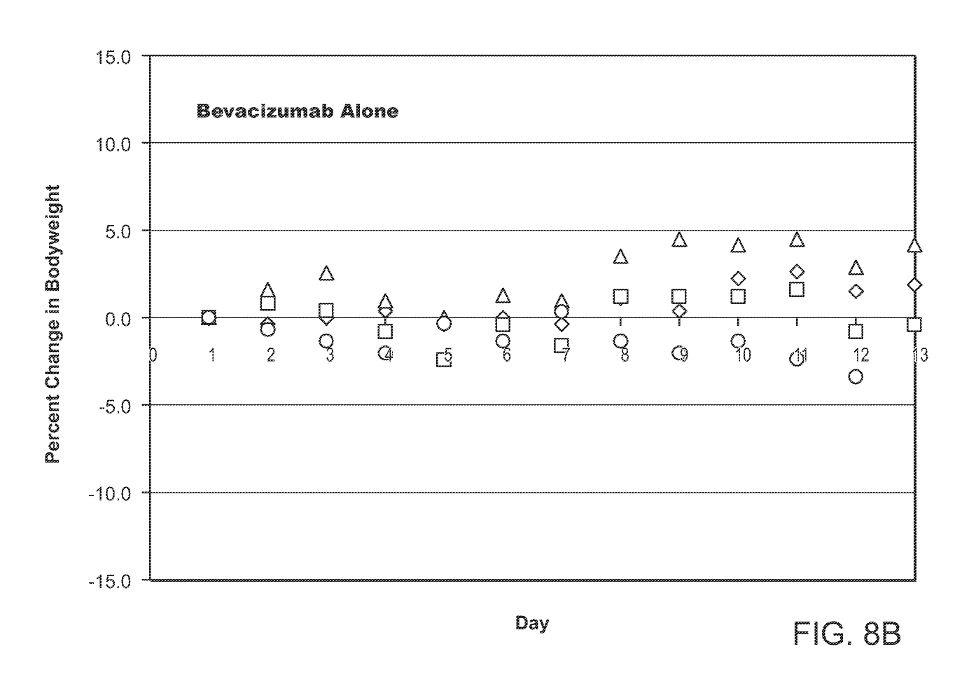

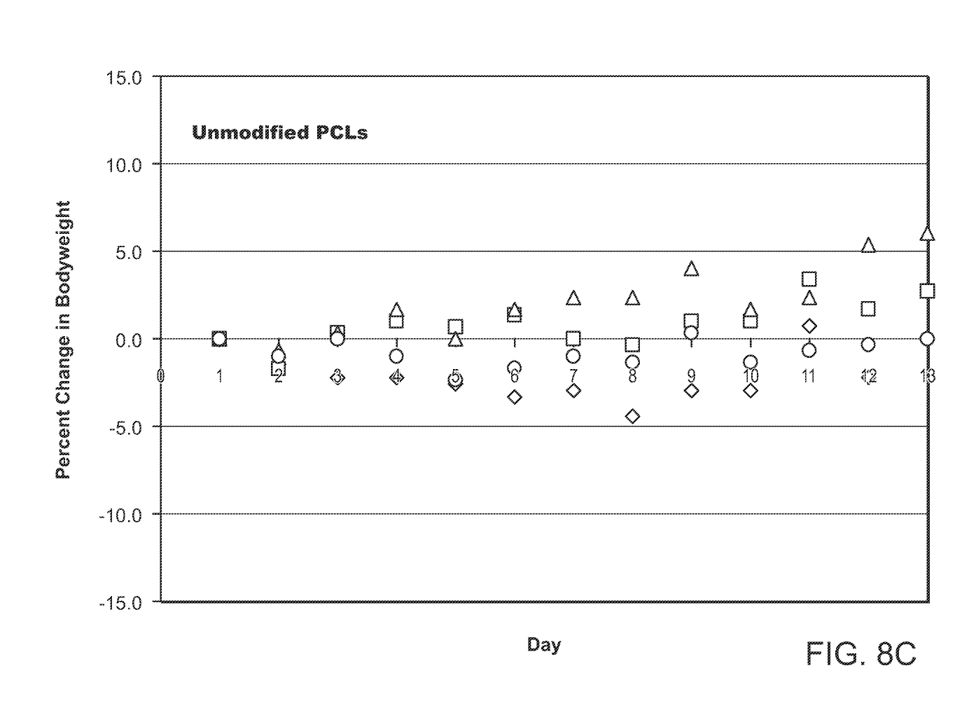

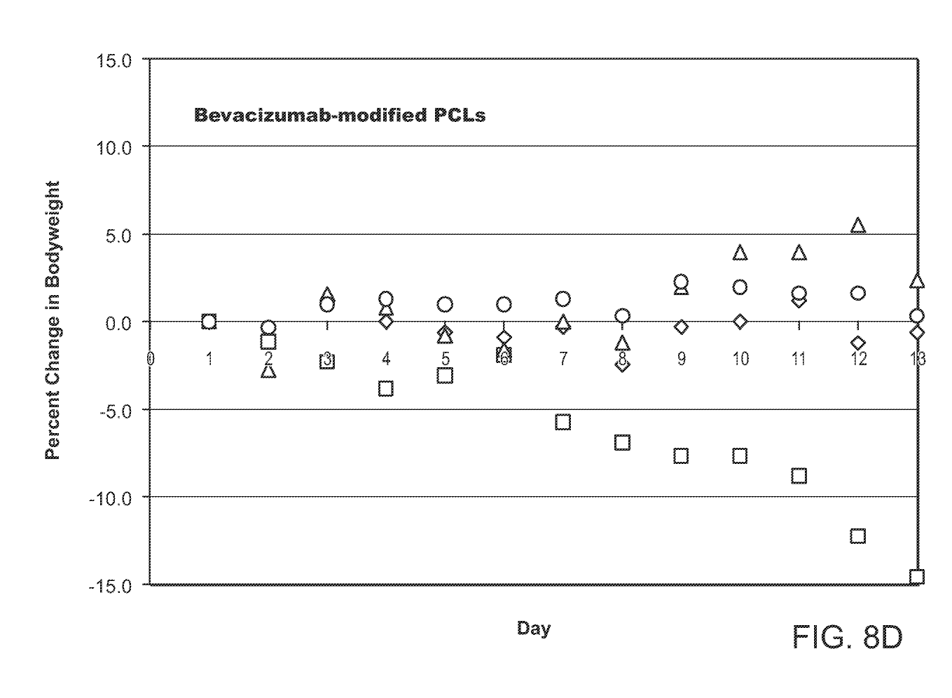

To assess therapeutic efficacy, four groups were evaluated, untreated control, 2-methoxyestradiol-loaded unmodified PCLs, bevacizumab alone and 2-methoxyestradiol-loaded bevacizumab-modified PCLs. The bevacizumab alone group was added to control for the ability of the antibody alone to inhibit tumor growth. None of the formulations was toxic to the mice at the doses given, as evidenced by the observation that the bodyweight did not decrease by more than 15% from the first day of injection (FIG. 8).

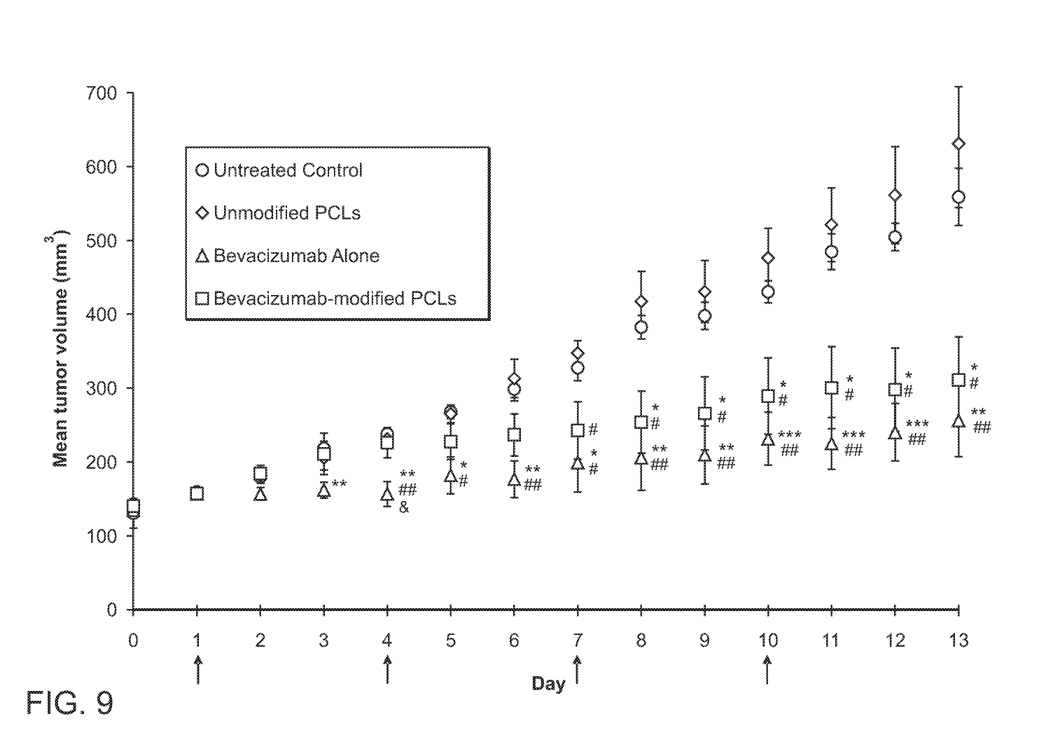

Tumor volumes were determined every day to evaluate tumor response to therapy. The 2-methoxyestradiol-loaded unmodified PCLs were ineffective at controlling tumor growth when compared to the untreated tumors. However, when both the untreated tumors and the tumors treated with unmodified PCLs were compared to the bevacizumab-modified PCLs, it was evident that the modified PCLs provided a significant benefit over the unmodified formulation at as early as day 7 and continued through the rest of the study. The bevacizumab-modified PCLs, though, were as effective as bevacizumab alone, where similar amounts of antibody were added between bevacizumab alone and the bevacizumab-modified PCLs (FIG. 9).

The response using bevacizumab alone was seen as early as day 3 and this response increased as the tumors in the control and unmodified PCL treated tumors continued to grow. One possible reason that the modified PCLs did not have a benefit over bevacizumab alone was that the dose of 2-methoxyestradiol given was not enough to be effective against this tumor. While not wishing to be bound by theory, it is believed that the bevacizumab-modified PCLs may have been effective using one of three possible mechanisms. First, the bevacizumab antibody attached to the PCLs was independently preventing tumor growth without the assistance of 2ME2. Second, the bevacizumab increased the targeting and uptake of the PCLs and thus increased 2ME2 uptake and drug effect. Third, is a combination of the two mechanisms.

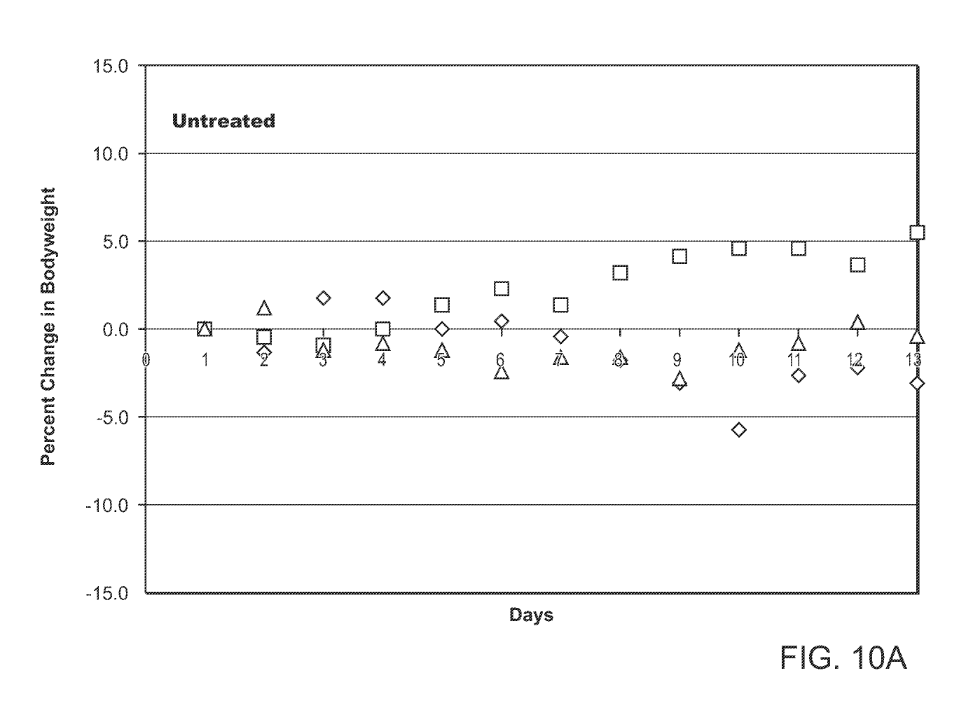

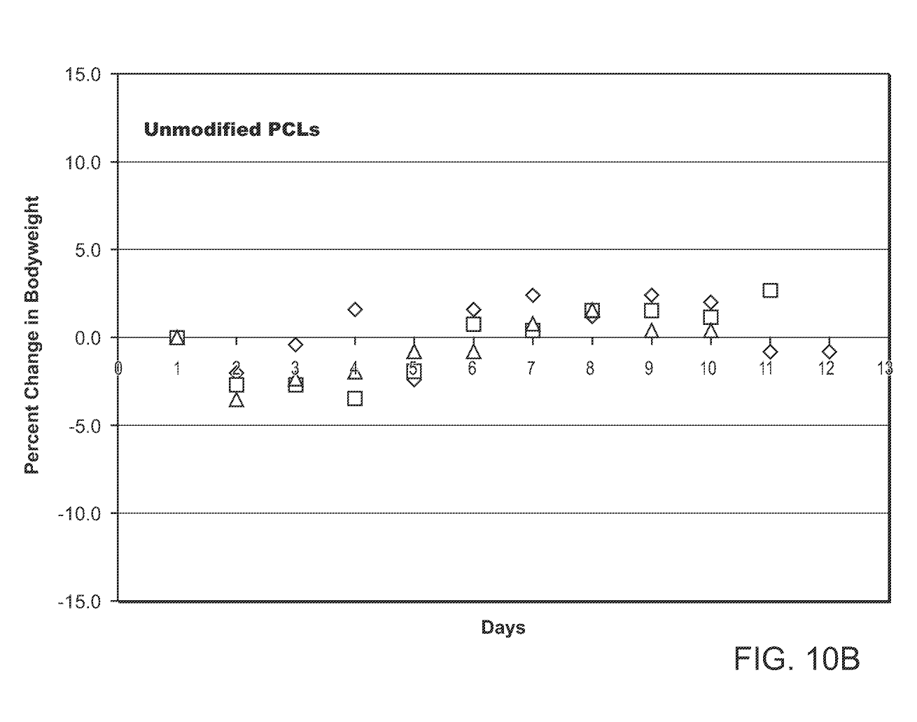

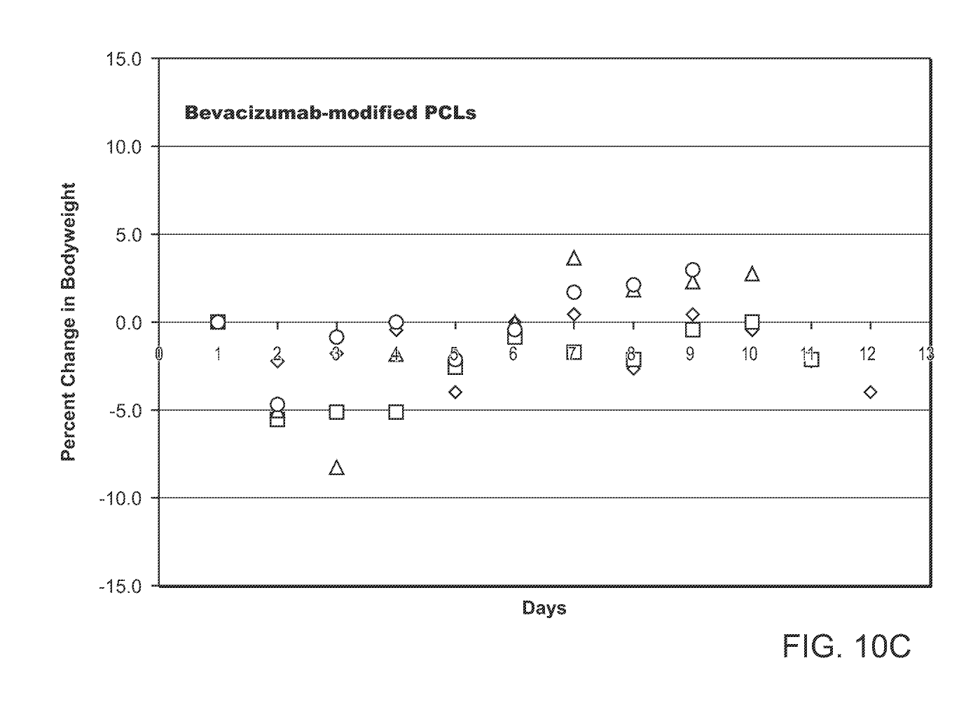

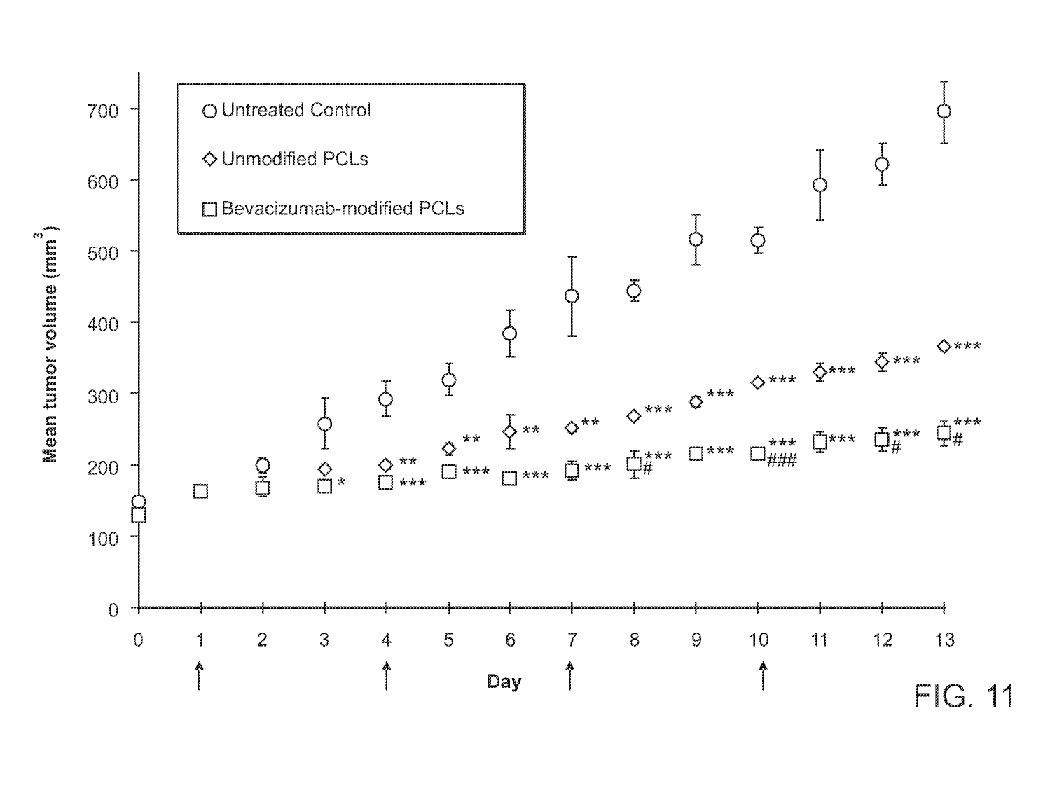

The second therapeutic study using a pancreatic tumor model with Capan-1 cells instead of HPAF-II cells differed only in that twice as many liposomes were injected, which contained in total twice as much 2-methoxyestradiol but the same amount of bevacizumab for the modified PCLs group. As can been seen by the bodyweight evaluation over the 13 days of evaluation, neither formulation was toxic to the mice even though twice as much lipid and drug were added (FIG. 10). In the Capan-1 tumor model, both the unmodified and bevacizumab-modified PCL formulations prevented significant growth when compared to the control (as early as day 3 for the modified PCLs and day 4 for the unmodified PCLs). As the study continued, the bevacizumab-modified PCLs became more effective at preventing tumor growth compared to the unmodified PCLs. While not wishing to be bound by theory, the antibody present on the surface of PCLs may provide direct therapeutic action by directly inhibiting VEGF. In addition, the conjugation of the antibody to the surface of PCLs may have improved the overall efficiency of targeting tumor vessels (or cells), such that significantly greater levels of 2-methoxyestradiol may have reached the tumor site (FIG. 11).

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of detecting a cancer cell in a subject, comprising:
(a) administering to the subject a liposome, the liposome comprising
(i) an anti-angiogenesis agent on an outer surface of the liposome, and (ii) a detection agent conjugated to the liposome; and
(b) detecting the detection agent, thereby detecting the cancer cell.

2. The method of claim 1, wherein the anti-angiogenesis agent is an anti-VEGF antibody.

3. The method of claim 2, wherein the anti-VEGF antibody is bevacizumab.

4. The method of claim 1, wherein the liposome comprises a cationic lipid.

5. The method of claim 4, wherein the cationic lipid is DDAB, DODAP, DOTAP, DOTMA, DMTAP, or DSTAP.

6. The method of claim 4, wherein the cationic lipid comprises a derivatized cationic lipid.

7. The method of claim 6, wherein the derivatized cationic lipid comprises polyethylene glycol (PEG).

8. The method of claim 1, wherein about 50% to about 100% of the outer surface of the liposome comprises the anti-angiogenesis agent.

9. The method of claim 1, wherein the anti-angiogenesis agent comprises about 20% to about 60% of the liposome by weight.

10. The method of claim 1, wherein the detection agent is a radionuclide.

11. A method of delivering a chemotherapeutic agent to a cancer cell, comprising contacting the cancer cell with a liposome, the liposome comprising:
(i) an anti-angiogenesis agent on an outer surface of the liposome, and (ii) a chemotherapeutic agent conjugated to the liposome,
the anti-angiogenesis agent targeting the cancer cell, thereby delivering the chemotherapeutic agent to the cancer cell.

12. The method of claim 11, wherein the anti-angiogenesis agent is an anti-VEGF antibody.

13. The method of claim 12, wherein the anti-VEGF antibody is bevacizumab.

14. The method of claim 11, wherein the liposome comprises a cationic lipid.

15. The method of claim 14, wherein the cationic lipid is DDAB, DODAP, DOTAP, DOTMA, DMTAP, or DSTAP.

16. The method of claim 14, wherein the cationic lipid comprises a derivatized cationic lipid.

17. The method of claim 16, wherein the derivatized cationic lipid comprises PEG.

18. The method of claim 11, wherein about 50% to about 100% of the outer surface of the liposome comprises the anti-angiogenesis agent.

19. The method of claim 11, wherein the anti-angiogenesis agent comprises about 20% to about 60% of the liposome by weight.

20. The method of claim 11, wherein the cancer cell is in a subject, and the chemotherapeutic agent is administered to the subject.

21. The method of claim 11, wherein the chemotherapeutic agent is delivered to the cell in vitro.

22. A method of treating a cancer cell in a subject, comprising administering to the subject a cationic liposome, the cationic liposome comprising:
(i) a cationic lipid;
(ii) PEG conjugated to the cationic lipid;
(iii) an anti-angiogenesis agent on an outer surface of the cationic liposome;
and
(iv) a chemotherapeutic agent conjugated to the liposome, the anti-angiogenesis agent targeting the cancer cell, thereby treating the cancer cell.

23. The method of claim 22, wherein the anti-angiogenesis agent is an anti-VEGF antibody.

24. The method of claim 23, wherein the anti-VEGF antibody is bevacizumab.
